(19) United States
(12) Patent Application Publication
(10) Pub. No.: US 2022/0275359 A1
(43) Pub. Date: Sep. 1, 2022

(54) GUIDE RNA FOR EDITING TARGET, WITH FUNCTIONAL NUCLEOTIDE SEQUENCE ADDED

(71) Applicants: Astellas Pharma Inc., Chuo-ku, Tokyo (JP); Fukuoka University, Fukuoka-shi, Fukuoka (JP)

(72) Inventors: Eiji YOSHIMI, Tokyo (JP); Yukari MORIYA, Tokyo (JP); Mariko MANDA, Tokyo (JP); Masatora FUKUDA, Fukuoka (JP)

(73) Assignees: Astellas Pharma Inc., Chuo-ku, Tokyo (JP); Fukuoka University, Fukuoka-shi, Fukuoka (JP)

(21) Appl. No.: 17/631,727

(22) PCT Filed: Jul. 31, 2020
(86) PCT No.: PCT/JP2020/029386

§ 371 Date: Jan. 31, 2022

(30) Foreign Application Priority Data
Aug. 1, 2019 (JP) 2019-141875

(51) Int. Cl. C12N 15/10; A61K 31/7105; C12N 5/10; C12N 15/11

(52) U.S. Cl. CPC C12N 15/102; A61K 31/7105; C12N 5/10; C12N 15/11; C12N 2310/11; C12N 2310/531

(57) ABSTRACT

[Problem to be Solved] To provide a guide RNA.

[Solution] A guide RNA for editing a target RNA sequence, comprising an antisense nucleotide sequence complementary to a portion of the target RNA sequence, a short-chain ADAR-recruiting nucleotide sequence, and at least one functional nucleotide sequence.

### TECHNICAL FIELD

The present invention relates to a guide RNA for editing a target RNA by inducing ADAR, and in particular, to a guide RNA having a functional nucleotide sequence added thereto.

### BACKGROUND ART

An RNA-editing mechanism involving a single nucleotide mutation is present in vivo. In particular, RNA editing of converting adenosine (A) to inosine (I) by deamination of the adenosine (A) is most frequently observed, and the number of targets reaches 4,000,000. ADAR (Adenosine deaminases acting on RNA) has been known as an enzyme of editing adenosine to inosine, using a double-stranded RNA as a substrate. ADAR has three different gene subtypes (ADAR1, ADAR2, and ADAR3), and all of these subtypes have a double-stranded RNA-binding domain at the N-terminus thereof and a deaminase domain at the C-terminus thereof.

ADAR2 edits with high efficiency, subunit 2 (GluR2) that constitutes a 3-amino-3-hydroxy-5-methyl-4-isoxazole propionic acid (AMPA)-type glutamic acid receptor, in particular, in the central nervous system (Nature, 1996, Vol. 379, pp. 460-464 (Non Patent Literature 1)).

Since inosine converted from adenosine has a structure similar to guanosine, the inosine is recognized as guanosine at the stage of translation, and it is substituted for an amino acid in a coding region. It has been reported that a retrotransposon as a non-coding region (FEBS Letters, 2006, Vol. 580, pp. 2301-2305) or a microRNA (miRNA) precursor becomes a substrate of ADAR, and it has been suggested that ADAR be likely to play various functions in vivo (Ann. Rev. Biochem., 2010, Vol. 79, pp. 321-349).

Modified ADAR has also been reported. For example, it has been reported that a mutation is inserted into ADAR, so that the modified ADAR is capable of recognizing not only adenosine but also cytidine, and as a result, RNA editing of converting C to U, in which cytidine is converted to uridine by deamination of the cytidine, can be performed (Science, 2019, Vol. 365, pp. 382-386).

In recent years, RNA editing using a wild-type ADAR and an artificial guide RNA has been reported.

For example, as an RNA editing technique using, as a guide RNA, an antisense oligonucleotide that is an artificial synthetic nucleic acid, there has been known and reported a technique of using a single-stranded RNA having a length of 35 or more nucleotides complementary to an mRNA containing adenosine as an editing target, and forming a double strand having an incomplete helix structure with the mRNA, so as to recruit ADAR (International Publication WO2017/220751, International Publication WO2018/041973, and International Publication WO2018/134301). Moreover, a target RNA-editing guide RNA consisting of an oligonucleotide construct of two regions, namely, a targeting region comprising an antisense sequence complementary to a portion of a target RNA and GluR2-derived ADAR-recruiting region, has been reported (Patent Literature 1). Furthermore, it has been reported that a guide RNA designed based on the sequence of GluR2 was allowed to express in a wild-type ADAR2-expressing cell line, so that certain RNA editing was induced (Nucleic Acids Research, 2017, Vol. 45, pp. 2797-2808 (Non Patent Literature 2), and Patent Literatures 2 and 3), and that an endogenous target was edited using a guide RNA constituted with a modified oligonucleotide (Nat. Biotechnol., 2019, Vol. 37, pp. 133-138 (Non Patent Literature 3)).

In addition, a site-specific target RNA-editing guide RNA, comprising an antisense region binding to a target RNA and an ADAR-recruiting region, wherein the ADAR-recruiting region has a length of 49 nucleotides or 40 nucleotides, has been reported (Scientific Reports, 2017, Vol. 7, p. 41478 (Non Patent Literature 4), and Patent Literature 4). Recently, a target-editing guide RNA comprising a first oligonucleotide specifying a target RNA and a second oligonucleotide having a length of 2 to 34 nucleotides that is connected with the 3′-terminal side of the first oligonucleotide has been reported (Patent Literature 5). According to Patent Literature 5, if the number of residues of the second oligonucleotide is 14 or more, it is considered that a stable stem-loop structure is formed, and that editing activity is maintained.

There are several reports regarding addition of a nucleotide sequence to an RNA such as a guide RNA.

For instance, it has been known that the stem-loop structure that is the higher-order structure of a nucleic acid found in small RNA contributes to a translational regulation function (Cell. Mol. LifeSci., 2006, Vol. 63, pp. 901-908). A BoxB sequence derived from λ phage has a stem-loop structure and exhibits a strong affinity with a λN protein (Biol. Cell., 2008, Vol. 100, pp. 125-138, J. Am. Chem. Soc., 2002, Vol. 124, pp. 10966-10967). It has been reported that, in order to allow a guide RNA to recruit ADAR by utilizing the aforementioned properties of the BoxB sequence, the BoxB sequence is added to the guide RNA, and the λN protein is fused with ADAR (Nucleic Acids Research, 2016, Vol. 44, p.e157 (Non Patent Literature 6)). Patent Literature 2 discloses that the BoxB sequence can be added to the 3′-terminus of a guide RNA in order to stabilize the guide RNA designed based on the sequence of GluR2. Meanwhile, Non Patent Literature 2 discloses that the stabilization effect obtained by addition of the BoxB sequence to the guide RNA was low.

A G-quadruplex (Gq) structure is a repeating sequence comprising guanine, which is known as a DNA or RNA higher-order structure that is thermodynamically stable in an in vivo environment. Monovalent cations are necessary for formation of a higher-order structure. Gq has been originally reported as a sequence found in a telomere (Cell, 1989, Vol. 59, pp. 871-880). Known Gq functions are transcription, translation, epigenome regulation, and the like (EMBO Reports, 2015, Vol. 16, pp. 910-922). It has been reported that the efficiency of genome editing is improved by adding Gq to the 3′-terminus of the guide RNA of Cas9 (Chem. Commun., 2018, Vol. 54, pp. 2377-2380 (Non Patent Literature 5)).

U6 small nuclear (sn) RNA is a small RNA that is stably expressed in a large amount in all human cells, and the snRNA forms the ribonucleoprotein U6 snRNP in the nucleus. U6 snRNP constitutes a spliceosome and regulates splicing in an RNA precursor. A transcription start element located upstream of U6 is known as a strong polIII-type promoter for expressing any given RNA (ribozyme, antisense ribonucleic acid, RNA aptamer, etc.) (Gene Ther., 1997, Vol. 4, pp. 45-54). It has been reported that, in the case of a small RNA expression cassette formed with a sequence encoded by U6, the higher-order structure of a U6 sequence functions to locate an RNA inserted into the expression cassette in the nucleus (Gene Ther., 1997, Vol. 4, pp. 45-54 (Non Patent Literature 7), Nat. Biotechnol., 2002, Vol. 20, pp. 505-508 (Non Patent Literature 8), and Mol. Ther., 2003, Vol. 7, pp. 237-247 (Non Patent Literature 9)).

In view of the foregoing, there are several reports regarding RNA editing using an artificial guide RNA. In order to use a target RNA-editing guide RNA as a pharmaceutical product, it is necessary for such a guide RNA to show certain editing efficiency in cells. However, the improvement of the editing efficiency of a guide RNA has not been sufficiently considered yet.

### CITATION LIST

### Patent Literature

Patent Literature 1: International Publication WO2016/097212
Patent Literature 2: International Publication WO2017/050306
Patent Literature 3: German Patent No. 102015012522
Patent Literature 4: International Publication WO2017/010556
Patent Literature 5: International Publication WO2019/111957

### Non Patent Literature

Non Patent Literature 1: Nature, 1996, Vol. 379, pp. 460-464
Non Patent Literature 2: Nucleic Acids Research, 2017, Vol. 45, pp. 2797-2808
Non Patent Literature 3: Nat. Biotechnol., 2019, Vol. 37, pp. 133-138
Non Patent Literature 4: Scientific Reports, 2017, Vol. 7, pp. 41478
Non Patent Literature 5: Chem. Commun., 2018, Vol. 54, pp. 2377-2380
Non Patent Literature 6: Nucleic Acids Research, 2016, Vol. 44, p. e157
Non Patent Literature 7: Gene Ther., 1997, Vol. 4, pp. 45-54
Non Patent Literature 8: Nat. Biotechnol., 2002, Vol. 20, pp. 505-508
Non Patent Literature 9: Mol. Ther., 2003, Vol. 7, pp. 237-247

### SUMMARY OF INVENTION

### Technical Problem

Provided are a guide RNA for editing a target RNA by inducing ADAR, and a nucleic acid encoding the same.

### Solution to Problem

The present inventors have conducted intensive studies regarding a guide RNA for editing a target RNA by inducing ADAR. As a result, the present inventors have found that a guide RNAhaving a functional nucleotide sequence added thereto shows high editing efficiency in cells, thereby completing the present invention.

Specifically, the present invention relates to the following [1] to [31].
[1] A guide RNA for editing a target RNA sequence, comprising: an antisense nucleotide sequence complementary to a portion of the target RNA sequence, a short-chain ADAR-recruiting nucleotide sequence, and at least one functional nucleotide sequence.
[2] The guide RNA according to the above [1], wherein the antisense nucleotide sequence, the short-chain ADAR-recruiting nucleotide sequence, and the at least one functional nucleotide sequence are connected with one another, in this order, from the 5′-terminal side to the 3′-terminal side.
[3] The guide RNA according to the above [2], wherein at least one functional nucleotide sequence is further connected with the 5′-terminal side of the antisense nucleotide sequence.
[4] The guide RNA according to the above [1], further comprising a linker nucleotide sequence.
[5] The guide RNA according to the above [4], wherein at least one functional nucleotide sequence is connected with the short-chain ADAR-recruiting nucleotide sequence via the linker nucleotide sequence.
[6] The guide RNA according to the above [5], wherein the antisense nucleotide sequence, the short-chain ADAR-recruiting nucleotide sequence, the linker nucleotide sequence, and the at least one functional nucleotide sequence are connected with one another, in this order, from the 5′-terminal side to the 3′-terminal side.
[7] The guide RNA according to the above [6], wherein at least one functional nucleotide sequence is further connected with the 5′-terminal side of the antisense nucleotide sequence.
[8] The guide RNA according to any one of the above [1] to [7], wherein the functional nucleotide sequence is a nucleotide sequence associated with the intracellular stabilization and/or intracellular localization of the guide RNA.
[9] The guide RNA according to any one of the above [1] to [8], wherein the functional nucleotide sequence(s) are one or more nucleotide sequences selected from a nucleotide sequence that forms a G-quadruplex structure and a nucleotide sequence that forms a stem-loop structure.
[10] The guide RNA according to any one of the above [1] to [8], wherein the functional nucleotide sequence is a U6 snRNA sequence.
[11] The guide RNA according to the above [9], wherein the functional nucleotide sequence is a nucleotide sequence that forms a 1 to 4 layered G-quadruplex structure.
[12] The guide RNA according to the above [9], wherein the functional nucleotide sequence is a nucleotide sequence that forms a stem-loop structure.
[13] The guide RNA according to the above [12], wherein the nucleotide sequence that forms a stem-loop structure is a nucleotide sequence derived from a BoxB sequence.
[14] The guide RNA according to any one of the above [1] to [13], wherein the functional nucleotide sequence consists of the nucleotide sequence as set forth in SEQ ID NO: 6, 7, 8, or 9, or a nucleotide sequence comprising a deletion, substitution, insertion and/or addition of 1 to 3 nucleotides with respect to the nucleotide sequence as set forth in SEQ ID NO: 6, 7, 8, or 9, and having functions.
[15] The guide RNA according to any one of the above [1] to [14], wherein the short-chain ADAR-recruiting nucleotide sequence is a nucleotide sequence consisting of 14 to 34 nucleotides.
[16] The guide RNA according to the above [15], wherein the short-chain ADAR-recruiting nucleotide sequence is a sequence consisting of 16 nucleotides.
[17] The guide RNA according to the above [15] or [16], wherein the loop portion in the stem-loop structure of the short-chain ADAR-recruiting nucleotide sequence is a nucleotide sequence consisting of 4 or 5 nucleotides.
[18] The guide RNA according to any one of the above [15] to [17], wherein the stem portion in the stem-loop structure of the short-chain ADAR-recruiting nucleotide sequence has nucleotides that form a mismatched base pair.
[19] The guide RNA according to any one of the above [1] to [18], wherein the short-chain ADAR-recruiting nucleotide sequence consists of the nucleotide sequence as set forth in SEQ ID NO: 1, 2, 3, or 4, or a nucleotide sequence comprising a deletion, substitution, insertion and/or addition of 1 to 3 nucleotides with respect to the nucleotide sequence as set forth in SEQ ID NO: 1, 2, 3, or 4, and forming a stem-loop structure.
[20] The guide RNA according to any one of the above [4] to [19], wherein the linker nucleotide sequence is a nucleotide sequence consisting of 15 to 25 nucleotides.
[21] A target RNA sequence-editing system, comprising the guide RNA according to any one of the above [1] to [20] and ADAR.
[22] A nucleic acid encoding the guide RNA according to any one of the above [1] to [20].
[23] An expression vector, comprising a nucleic acid encoding the guide RNA according to any one of the above [1] to [20].
[24] The expression vector according to the above [23], further comprising a nucleic acid encoding ADAR.
[25] A host cell, into which the expression vector according to the above [23] or [24] is introduced.
[26] A method for producing an expression vector, comprising a step of culturing the host cell according to the above [25].
[27] A pharmaceutical composition, comprising the expression vector according to the above [23] or [24] and a pharmaceutically acceptable excipient.
[28] A pharmaceutical composition, comprising the expression vector according to the above [23], an expression vector containing a nucleic acid encoding ADAR, and a pharmaceutically acceptable excipient.
[29] The pharmaceutical composition according to the above [27] or [28], wherein the ADAR is human ADAR1 or human ADAR2.
[30] The pharmaceutical composition according to the above [28] or [29], wherein the nucleic acid encoding ADAR is a nucleic acid consisting of a nucleotide sequence encoding the amino acid sequence as set forth in SEQ ID NO: 12, or a nucleotide sequence encoding a polypeptide that consists of an amino acid sequence having an identity of 90% or more to the amino acid sequence as set forth in SEQ ID NO: 12 and has an adenosine deaminase activity.
[31] A method for editing a target RNA, comprising: (i) a step of introducing the guide RNA according to any one of the above [1] to [20] or the nucleic acid according to the above [22] into a cell; (ii) a step of recruiting ADAR by the guide RNA; (iii) a step of forming a complex of a target RNA sequence and the guide RNA; (iv) a step of converting the adenosine of the target RNA sequence to inosine by the ADAR recruited by the guide RNA.

### Advantageous Effects of Invention

A guide RNA having a functional nucleotide sequence added thereto can be used as a guide RNA for editing a target RNA in a cell.

### BRIEF DESCRIPTION OF DRAWINGS

FIG. 1 is a schematic view showing the guide RNA of the present invention.

FIG. 2 shows representative examples of the two times experiments of luciferase assays (RNA-editing activity 3 days after the transfection). The longitudinal axis indicates a relative value of the Rluc/Fluc value of each guide RNA, when the Rluc/Fluc value of a guide RNA used as a control is set to be 1. The horizontal axis indicates the numbers (#) of plasmids. Besides, the experiments were each carried out for every 3 wells. The graph is shown as an arithmetic mean of the 3 wells and standard deviation.

FIG. 3 shows representative examples of the two times experiments of luciferase assays (RNA-editing activity 6 days after the transfection). The longitudinal axis indicates a relative value of the Rluc/Fluc value of each guide RNA, when the Rluc/Fluc value of a guide RNA used as a control is set to be 1. The horizontal axis indicates the numbers (#) of plasmids. Besides, the experiments were each carried out for every 3 wells. The graph is shown as an arithmetic mean of the 3 wells and standard deviation.

FIG. 4 shows the Rluc/Fluc value (RNA-editing activity 3 days after the transfection) obtained by luciferase assay. The longitudinal axis indicates the Rluc/Fluc value. The horizontal axis indicates the numbers (#) of plasmids. Besides, the experiment was carried out three times. The graph is shown as an arithmetic mean of the 3 trials and standard deviation. The plot indicates an arithmetic mean of every 3 samples in each trial.

### DESCRIPTION OF EMBODIMENTS

The present invention will be described in detail below. However, the present invention is not limited to the following explanation. The terms used in the present description have meanings generally accepted by those skilled in the art, unless otherwise specifically defined.

### <Outline>

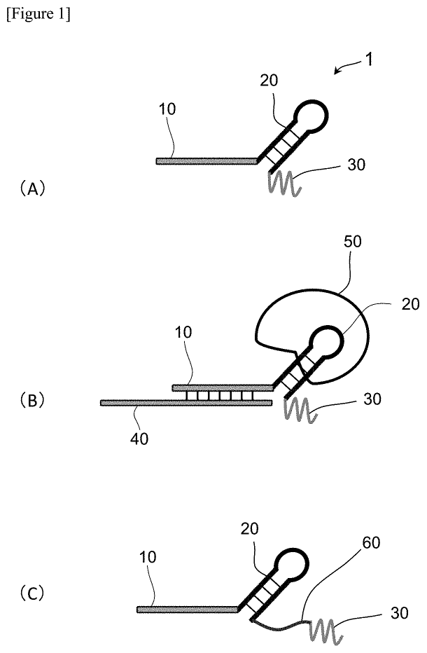

The present invention relates to a guide RNA for editing a target RNA sequence. According to RNA editing, for example, a specific nucleotide in a target sequence is substituted with another nucleotide, so that the function or expression of a protein encoded by the target sequence can be regulated (for example, can be enhanced or decreased). FIG. 1 is a schematic view of the guide RNA (1) of the present invention, and as shown in FIG. 1A, the basic structure of the guide RNA (1) is composed of an antisense nucleotide sequence (10) complementary to a portion of a target RNA sequence, a short-chain ADAR-recruiting nucleotide sequence (20), and at least one functional nucleotide sequence (30).

The antisense nucleotide sequence (10) complementary to a portion of the target RNA sequence has a sequence complementary to the sequence of a partial region of the target RNA sequence. Thus, as shown in FIG. 1B, the above-described antisense nucleotide sequence (10) can hybridize to a target RNA sequence (40). The short-chain ADAR-recruiting nucleotide sequence (20) is a nucleotide sequence that induces (recruits) an ADAR (50) and forms a complex with the ADAR (50). According to the thus recruited ADAR, a nucleotide in the target RNA sequence can be substituted with another nucleotide. The at least one functional nucleotide sequence (30) imparts any function such as intracellular stabilization or intracellular localization to the guide RNA.

As shown in FIG. 1C, in the present invention, a linker (60) is further added to the above-described sequence, so that the guide RNA of the present invention can more effectively enhance the function of ADAR in cells.

FIG. 1 is an illustrative example for showing a basic concept of the guide RNA of the present invention, and thus, the present invention is not limited to this aspect.

### <Guide RNA of the Present Invention>

In the present invention, a guide RNA for editing a target RNA sequence (hereinafter also referred to as “the guide RNA of the present invention”) means an RNA molecule that binds to ADAR and recruits the ADAR to a target RNA sequence. The guide RNA for editing a target RNA sequence of the present invention comprises an antisense nucleotide sequence complementary to a portion of the target RNA sequence, a short-chain ADAR-recruiting nucleotide sequence, and at least one functional nucleotide sequence. In a certain aspect, the guide RNA for editing a target RNA sequence of the present invention comprises an antisense nucleotide sequence complementary to a portion of the target RNA sequence, a short-chain ADAR-recruiting nucleotide sequence, a linker nucleotide sequence, and at least one functional nucleotide sequence.

In the present invention, the RNA may include an RNA comprising a modified nucleic acid and/or a nucleic acid analog. The modification that can be used in the present invention is, for example, nucleotide modification or skeletal modification, and such modification is well known to those skilled in the art.

Examples of a modified nucleic acid, which is used in the present invention, may include modified nucleotides such as 2′-O-alkyl ribose, 2′-O-methyl ribose, 2′-fluororibose, phosphorothioate, methylphosphonate, a phosphoramidite-morpholino oligomer (PMO), 5-methyl-dC, 2-amino-dA, and C5-pyrimidine. A preferred example of a stable modified nucleotide is a 2′-O-methyl modified nucleotide.

Examples of a nucleic acid analog used in the present invention may include a locked nucleic acid (LNA) nucleotide and/or a peptide nucleic acid (PNA).

In addition, in the present invention, the guide RNA may comprise a phosphorothioate bond between nucleotides.

### 1. Target RNA Sequence

In the present invention, the “target RNA sequence” means an RNA sequence as a target of the editing according to the guide RNA of the present invention. In a certain aspect, the target RNA sequence is any given RNA sequence that comprises adenosine, wherein the adenosine is converted (edited) to inosine, so that the target RNA sequence can regulate cell function or gene expression. In another aspect, the target RNA sequence is any given RNA sequence that comprises cytidine, wherein the cytidine is converted (edited) to uridine, so that the target RNA sequence can regulate cell function or gene expression. The target RNA sequence can be present in an exon, an intron, a coding region in the exon, or various types of untranslated regions such as, for example, retrotransposon, or an RNA including a microRNA (miRNA), a transfer RNA (tRNA), and a ribosomal RNA (rRNA). The target RNA sequence can also be present in an RNA derived from virus.

### 2. Antisense Nucleotide Sequence

The guide RNA of the present invention comprises an antisense nucleotide sequence complementary to a portion of the target RNA sequence (which is simply referred to as an “antisense nucleotide sequence” at times). In the present invention, the “antisense nucleotide sequence” is a sequence that has a nucleotide sequence complementary to a portion of the target RNA sequence and forms a double strand with the target RNA sequence. The length of the antisense nucleotide sequence is, in a certain aspect, 10 nucleotides to 100 nucleotides, 10 nucleotides to 80 nucleotides, 10 nucleotides to 60 nucleotides, 10 nucleotides to 40 nucleotides, or 10 nucleotides to 30 nucleotides, in a certain aspect, 15 to 25 nucleotides, in a certain aspect, 18 to 25 nucleotides, in a certain aspect, 35 nucleotides to 40 nucleotides, and in a certain aspect, 70 nucleotides to 100 nucleotides. In a certain aspect, the antisense nucleotide sequence may comprise a nucleotide that forms a mismatched base pair with the target RNA sequence, or a nucleotide that forms a wobble base pair with the target RNA sequence.

The “mismatched base pairs” in the present description are the base pairs G-A, C-A, U-C, A-A, G-G, C-C, and U-U. The “wobble base pairs” in the present description are the base pairs G-U, I-U, I-A, and I-C.

The antisense nucleotide sequence is, in a certain aspect, a nucleotide sequence consisting of 18 to 25 nucleotides that has a nucleotide forming a mismatched base pair with adenosine of the target RNA and forms a complementary base pair with the target RNA.

The antisense nucleotide sequence can be designed, as appropriate, by those skilled in the art, while taking into consideration the sequence of the target RNA, the base length thereof, the position of a mismatched base pair formed with adenosine or cytidine of the target RNA, off-target effect, etc.

### 3. Short-Chain ADAR-Recruiting Nucleotide Sequence

In the present invention, the “ADAR-recruiting nucleotide sequence” is a nucleotide sequence that recruits ADAR and forms a stem-loop structure. The “short-chain ADAR-recruiting nucleotide sequence” is a short ADAR-recruiting nucleotide sequence, compared with an ADAR-recruiting nucleotide sequence consisting of 40 nucleotides (ARR(40), SEQ ID NO: 5) produced from an ADAR-recruiting nucleotide sequence consisting of 49 nucleotides derived from GluR2 (Patent Literature 4). The length of the short-chain ADAR-recruiting nucleotide sequence is, for example, 14 to 34 nucleotides, in a certain aspect, 14, 16, 24, or 34 nucleotides, in a certain aspect, 14 or 16 nucleotides, and in another aspect, 16 nucleotides.

In the present description, the phrase “to recruit ADAR” means that the nucleotide sequence binds to ADAR and induces the ADAR to the target RNA. The short-chain ADAR-recruiting nucleotide sequence of the present invention is not particularly limited, as long as it forms a structure that binds to ADAR. The stem-loop structure is also referred to as a hairpin structure, and it is publicly known in the present technical field. As known in the present technical field, since the stem-loop structure does not need the formation of a completely complementary base pair, the stem structure portion may comprise one or more mismatched base pairs or wobble base pairs. In a certain aspect, the short-chain ADAR-recruiting nucleotide sequence is a nucleotide sequence having a nucleotide that forms a mismatched base pair or a wobble base pair in a double-stranded RNA portion that forms a stem, and forming a stem-loop structure.

The short-chain ADAR-recruiting nucleotide sequence can be designed based on, for example, the stem-loop structure of the mRNA precursor of GluR2 (Patent Literature 4 and Non Patent Literature 4). In a certain aspect, the short-chain ADAR-recruiting nucleotide sequence can also be produced by shortening the stem portion of a nucleotide sequence forming the stem-loop structure of the mRNA precursor of GluR2, wherein the nucleotide sequence recruits ADAR, while retaining the recruiting function thereof.

In a certain aspect, the short-chain ADAR-recruiting nucleotide sequence may be a nucleotide sequence, in which the nucleotide sequence of a loop portion in the aforementioned stem portion-shortened nucleotide sequence is replaced with another nucleotide sequence that forms a loop. In a certain aspect, the short-chain ADAR-recruiting nucleotide sequence may be derived from an ADAR substrate other than the mRNA precursor of GluR2. Such an ADAR substrate that can be used in the designing of the ADAR-recruiting nucleotide sequence is well known to those skilled in the art (Biochemistry, 2018, Vol. 57, pp. 1640-1651, RNA, 2011, Vol. 2, pp. 761-771).

The nucleotide sequence of the loop structure portion of the short-chain ADAR-recruiting nucleotide sequence is not particularly limited, as long as it is a sequence that stably maintains a loop structure, from the viewpoint of nucleic acid chemistry. In a certain aspect, the nucleotide sequence of the loop structure portion is a nucleotide sequence consisting of GCUMA that is systematically conserved in GluR2 (wherein M represents A or C), and in a certain aspect, it is a nucleotide sequence consisting of GCUAA (Biophysical Chemistry, 2013, Vol. 180-181, pp. 110-118). In a certain aspect, the nucleotide sequence of the loop structure portion may be a nucleotide sequence that forms a tetraloop fold, and in another aspect, it is UUCG (ACS Chemical Biology, 2006, Vol. 1, pp. 761-765, Biophysical J., 2017, Vol. 113, pp. 257-267). The length of the loop structure portion is, in a certain aspect, 5 nucleotides, and it is, in a certain aspect, 4 nucleotides.

In a certain aspect, the short-chain ADAR-recruiting nucleotide sequence consists of the nucleotide sequence as set forth in SEQ ID NO: 1 (ARR(14)), or a nucleotide sequence having an identity of 90% or more to the nucleotide sequence as set forth in SEQ ID NO: 1, and forming a stem-loop structure, or a nucleotide sequence comprising a deletion, substitution, insertion and/or addition of 1 or 2 nucleotides with respect to the nucleotide sequence as set forth in SEQ ID NO: 1, and forming a stem-loop structure. In a certain aspect, the short-chain ADAR-recruiting nucleotide sequence consists of the nucleotide sequence as set forth in SEQ ID NO: 2 (ARR(16)), or a nucleotide sequence having an identity of 90% or more to the nucleotide sequence as set forth in SEQ ID NO: 2, and forming a stem-loop structure, or a nucleotide sequence comprising a deletion, substitution, insertion and/or addition of 1 or 2 nucleotides with respect to the nucleotide sequence as set forth in SEQ ID NO: 2, and forming a stem-loop structure. In a certain aspect, the short-chain ADAR-recruiting nucleotide sequence consists of the nucleotide sequence as set forth in SEQ ID NO: 3 (ARR(24)), or a nucleotide sequence having an identity of 90% or more to the nucleotide sequence as set forth in SEQ ID NO: 3, and forming a stem-loop structure, or a nucleotide sequence comprising a deletion, substitution, insertion and/or addition of 1 to 3 nucleotides with respect to the nucleotide sequence as set forth in SEQ ID NO: 3, and forming a stem-loop structure. In a certain aspect, the short-chain ADAR-recruiting nucleotide sequence consists of the nucleotide sequence as set forth in SEQ ID NO: 4 (ARR(34)), or a nucleotide sequence having an identity of 90% or more to the nucleotide sequence as set forth in SEQ ID NO: 4, and forming a stem-loop structure, or a nucleotide sequence comprising a deletion, substitution, insertion and/or addition of 1 to 4 nucleotides with respect to the nucleotide sequence as set forth in SEQ ID NO: 4, and forming a stem-loop structure.

In the present description, the term “ARR” means an ADAR-recruiting nucleotide sequence (ADAR Recruiting Region). The number in the parentheses after ARR indicates the base length of the ARR, and for example, “ARR(14)” means an ADAR-recruiting nucleotide sequence having a length of 14 nucleotides. The term “ASR” means an antisense nucleotide sequence (Antisense Region).

In the present description, the term “identity” means Identity that is a value obtained using parameters set to default values according to the EMBOSS NEEDLE program (J. Mol. Biol., 1970, Vol. 48, pp. 443-453) searching. The above-described parameters are as follows.

Gap Open penalty =10

Gap Extend penalty =0.5

Matrix =EBLOSUM62

4. Linker Nucleotide Sequence

The guide RNA of the present invention may comprise a linker nucleotide sequence. For example, by allowing the present guide RNA to comprise such a linker nucleotide sequence, the function or efficiency of the after-mentioned functional nucleotide sequence can be improved.

The linker nucleotide sequence means a nucleotide sequence that connects a certain nucleotide sequence with another nucleotide sequence, and the linker nucleotide sequence may be, for example, a nucleotide sequence that connects the short-chain ADAR-recruiting nucleotide sequence with a functional nucleotide sequence. In a certain aspect, the guide RNA of the present invention comprises one or more linker nucleotide sequences selected from among a linker nucleotide sequence that connects the short-chain ADAR-recruiting nucleotide sequence with a functional nucleotide sequence, a linker nucleotide sequence that connects the functional nucleotide sequence with the antisense nucleotide sequence, and a linker nucleotide sequence that connects a plurality of functional nucleotide sequences with one another.

The linker nucleotide sequence is, in a certain aspect, a nucleotide sequence that does not form a base pair with the target RNA sequence. The linker nucleotide sequence is, in a certain aspect, a nucleotide sequence that does not interact with the short-chain ADAR-recruiting nucleotide sequence. The linker nucleotide sequence is, in a certain aspect, a nucleotide sequence that neither forms a base pair with the target RNA sequence, nor interacts with the short-chain ADAR-recruiting nucleotide sequence. The linker nucleotide sequence is, in a certain aspect, a nucleotide sequence that forms a stem-loop structure in the linker nucleotide sequence itself. The linker nucleotide sequence is, in a certain aspect, a nucleotide sequence having moderate heat stability. The linker nucleotide sequence is, in a certain aspect, a nucleotide sequence that can immobilize the antisense nucleotide sequence and the short-chain ADAR-recruiting nucleotide sequence, to such an extent that ADAR can be stably induced to the target RNA sequence. The linker nucleotide sequence can be designed, as appropriate, by those skilled in the art, based on the sequence that constitutes the guide RNA. For example, the linker nucleotide sequence used in the present invention may be designed such that it does not form a complementary strand with the target RNA sequence. Otherwise, it is also possible to design the linker nucleotide sequence, such that a small loop is formed with 4 or 5 nucleotides.

The length of such a linker nucleotide sequence is, in a certain aspect, 15 to 25 nucleotides, and it is, in a certain aspect, 20 nucleotides. In a certain aspect, the linker nucleotide sequence consists of the nucleotide sequence as set forth in SEQ ID NO: 10 (add(20)), or a nucleotide sequence having an identity of 90% or more to the nucleotide sequence as set forth in SEQ ID NO: 10, or a nucleotide sequence comprising a deletion, substitution, insertion and/or addition of 1 or 2 nucleotides with respect to the nucleotide sequence as set forth in SEQ ID NO: 10.

In the present description, the term “add” means a linker nucleotide sequence.

In a certain aspect, the linker nucleotide sequence is a short linker nucleotide sequence (14 or less nucleotides). In a certain aspect, the guide RNA of the present invention may comprise a short linker nucleotide sequence, between the functional nucleotide sequence and the antisense nucleotide sequence, between the short-chain ADAR-recruiting nucleotide sequence and the functional nucleotide sequence, and/or between two or more of the functional nucleotide sequences. In a certain aspect, the guide RNA of the present invention may comprise a linker nucleotide sequence between the short-chain ADAR-recruiting nucleotide sequence and the functional nucleotide sequence, and may further comprise another short linker nucleotide sequence between the functional nucleotide sequence and the antisense nucleotide sequence, between the aforementioned linker nucleotide sequence and the functional nucleotide sequence, and/or between two or more of the functional nucleotide sequences. In a certain aspect, such a short linker nucleotide sequence consists of a nucleotide sequence that does not form a stem-loop structure in each single molecule. In a certain aspect, such a short linker nucleotide sequence consists of a nucleotide sequence that does not form a complementary strand with the target RNA. In a certain aspect, the length of such a short linker nucleotide sequence is 1 to 10 nucleotides, and it is 1 to 5 nucleotides in a certain aspect, 1 to 3 nucleotides in a certain aspect, 3 to 5 nucleotides in a certain aspect, and 3 nucleotides in a certain aspect. In a certain aspect, the short linker nucleotide sequence is “UCU.” The short linker nucleotide sequence can be designed, as appropriate, by those skilled in the art, based on the sequence that constitutes the guide RNA. For example, the short linker nucleotide sequence used in the present invention may be designed, such that it does not form a complementary strand with the target RNA and it does not form a stem-loop structure in each single molecule of the linker.

### 5. Functional Nucleotide Sequence

The guide RNA of the present invention comprises at least one functional nucleotide sequence. In a certain aspect, the guide RNA of the present invention may comprise at least two functional nucleotide sequences of the same species. In another aspect, the guide RNA of the present invention may comprise at least two functional nucleotide sequences of two or more different species.

In the present invention, the “functional nucleotide sequence” means a nucleotide sequence that can impart to the guide RNA, any function such as the intracellular stabilization and/or intracellular localization of the guide RNA. Examples of the functional nucleotide sequence may include a nucleotide sequence that promotes the intracellular stabilization of the guide RNA, a nucleotide sequence that promotes localization of the guide RNA into the nucleus, and a nucleotide sequence including both of the two functions. In a certain aspect, the guide RNA of the present invention comprises, as such a functional nucleotide sequence, a nucleotide sequence that promotes the intracellular stabilization of the guide RNA, or a nucleotide sequence that promotes localization of the guide RNA into the nucleus, or both of the two nucleotide sequences. In a certain aspect, the nucleotide sequence that promotes the intracellular stabilization of the guide RNA includes a nucleotide sequence that forms a thermodynamically stabilizing higher-order structure. In a certain aspect, the nucleotide sequence that promotes localization of the guide RNA into the nucleus includes a nucleotide sequence that is derived from a small RNA (low molecular RNA) and promotes localization of the guide RNA into the nucleus. In a certain aspect, the functional nucleotide sequence includes a nucleotide sequence that forms a thermodynamically stabilizing higher-order structure and/or a nucleotide sequence that is derived from a small RNA and promotes localization of the guide RNA into the nucleus.

In a certain aspect, the guide RNA of the present invention comprises, as a functional nucleotide sequence(s), a nucleotide sequence that forms a thermodynamically stabilizing higher-order structure and/or a nucleotide sequence derived from a small RNA. The thermodynamically stabilizing higher-order structure is not particularly limited, as long as it is a structure that suppresses the decomposition of the guide RNA by nuclease. Examples of the thermodynamically stabilizing higher-order structure may include a double strand, a triple strand, a quadruple strand, and a stem-loop structure. In a certain aspect, examples of the functional nucleotide sequence may include a nucleotide sequence that forms a G-quadruplex (Gq) structure (Gq sequence) and/or a nucleotide sequence that forms a stem-loop structure. In a certain aspect, the guide RNA of the present invention comprises one or more functional nucleotide sequences selected from a Gq sequence and a nucleotide sequence that forms a stem-loop structure. The common structure of the Gq sequence is well known to those skilled in the art. The stem-loop structure is also referred to as a “hairpin structure,” and is publicly known in the present technical field. Examples of the nucleotide sequence that forms a stem-loop structure may include an aptamer sequence (Int. J. Biochem. Mol. Biol., 2013, Vol. 4, pp. 27-40), a nucleotide sequence derived from a BoxB sequence, a nucleotide sequence derived from an MS2 sequence, and a nucleotide sequence derived from a PP7 sequence (Integr. Biol., 2009, Vol. 1, pp. 499-505, Nucleic Acids Research, 2016, Vol. 44, pp. 9555-9564). In a certain aspect, the guide RNA of the present invention comprises, as a functional nucleotide sequence, a nucleotide sequence derived from a BoxB sequence. In a certain aspect, the nucleotide sequence derived from a small RNA includes a U6 small nuclear (sn) RNA sequence.

The functional nucleotide sequence used in the present invention is, in a certain aspect, a Gq sequence, a U6 snRNA sequence, a BoxB sequence-derived nucleotide sequence, an MS2-derived nucleotide sequence, a PP7-derived nucleotide sequence, or a combination thereof. In a certain aspect, the functional nucleotide sequence used in the present invention is a Gq sequence, a U6 snRNA sequence, and/or a BoxB sequence-derived nucleotide sequence, or a combination thereof.

The Gq sequence used in the present invention is a sequence comprising 4 repeating units consisting of contiguous guanine residues, and 4 guanine residues form a planar quadruple strand (Gq) structure. The repeating units consisting of guanine residues may comprise nucleotides other than guanine among the units, as long as they maintain the Gq structure. In a certain aspect, the Gq sequence comprises repeating units each consisting of 1, 2, 3, or 4 guanine residues, and the repeating units each form 1, 2, 3, or 4 layers of Gq. In a certain aspect, the Gq sequence comprises repeating units each consisting of 3 guanine residues, and the repeating units form 3 layers of Gq. This is referred to as a “3 layered G-quadruplex structure” (which is also referred to as “3Gq” in the present description). In a certain aspect, the guide RNA of the present invention comprises 3Gq as a functional nucleotide sequence, and in a certain aspect, the nucleotide sequence that forms 3Gq consists of the nucleotide sequence as set forth in SEQ ID NO: 6, or a nucleotide sequence comprising a deletion, substitution, insertion and/or addition of 1 to 3 nucleotides with respect to the nucleotide sequence as set forth in SEQ ID NO: 6, and forming a Gq structure.

The U6 snRNA sequence used in the present invention is a sequence that is constituted with the nucleotide sequence of U6 snRNA. In the guide RNA of the present invention, the U6 snRNA sequence may be connected with the short-chain ADAR-recruiting nucleotide sequence and/or the antisense nucleotide sequence and may be then used, as long as it has the function of promoting localization of the guide RNA into the nucleus. Otherwise, the short-chain ADAR-recruiting nucleotide sequence and the antisense nucleotide sequence may be inserted into the U6 snRNA sequence, and may be then used. When the antisense nucleotide sequence and the short-chain ADAR-recruiting nucleotide sequence may be inserted into the U6 snRNA sequence and may be then used, the U6 snRNA sequence is divided into any given two regions (wherein the 5′-terminal side is referred to as an “upstream region”, whereas the 3′-terminal side is referred to as a “downstream region”), and thereafter, the antisense nucleotide sequence and the short-chain ADAR-recruiting nucleotide sequence are inserted between the two regions and are then used. In a certain aspect of the guide RNA of the present invention comprising the U6 snRNA sequence, the antisense nucleotide sequence and the ADAR-recruiting nucleotide sequence are located between the upstream region of the U6 snRNA sequence and the downstream region of the U6 snRNA sequence, and thus, the upstream region of the U6 snRNA sequence, the antisense nucleotide sequence and the ADAR-recruiting nucleotide sequence, and the downstream region of the U6 snRNA sequence are connected with one another, in this order, from the 5′-terminal side to the 3′-terminal side. In a certain aspect, the nucleotide sequence of the upstream region of the U6 snRNA sequence used in the present invention consists of the nucleotide sequence as set forth in SEQ ID NO: 8 (also referred to as “U6 upstream”), or a nucleotide sequence comprising a deletion, substitution, insertion and/or addition of 1 to 3 nucleotides with respect to the nucleotide sequence as set forth in SEQ ID NO: 8, and having the function of promoting localization of the guide RNA into the nucleus when the nucleotide sequence is used together with the sequence of the downstream region of the U6 snRNA sequence. In a certain aspect, the nucleotide sequence of the downstream region of the U6 snRNA sequence used in the present invention consists of the nucleotide sequence as set forth in SEQ ID NO: 9 (also referred to as “U6 downstream”), or a nucleotide sequence comprising a deletion, substitution, insertion and/or addition of 1 to 3 nucleotides with respect to the nucleotide sequence as set forth in SEQ ID NO: 9, and having the function of promoting localization of the guide RNA into the nucleus when the nucleotide sequence is used together with the sequence of the upstream region of the U6 snRNA sequence.

In a certain aspect, the BoxB sequence-derived sequence used in the present invention consists of the nucleotide sequence as set forth in SEQ ID NO: 7 (which is also referred to as “BoxB”), or a nucleotide sequence having an identity of 90% or more to the nucleotide sequence as set forth in SEQ ID NO: 7, and forming a stem-loop structure, or a nucleotide sequence comprising a deletion, substitution, insertion and/or addition of 1 to 3 nucleotides with respect to the nucleotide sequence as set forth in SEQ ID NO: 7, and forming a stem-loop structure.

With regard to the functional nucleotide sequence, what the functional nucleotide sequence forms a Gq structure, forms a stem-loop structure, and has the function of promoting localization of the guide RNA into the nucleus when it is used together with the sequence of the upstream region or downstream region of the U6 snRNA sequence, is collectively referred to as “having functions,” at times.

The functions of the functional nucleotide sequence to the guide RNA can be confirmed by known methods. Stabilization of the guide RNA can be confirmed, for example, by the methods described in Reference Examples 1 and 2, although the methods are not particularly limited thereto. It is considered that such stabilization of the guide RNA can reduce the dose of a pharmaceutical composition comprising the guide RNA, a nucleic acid encoding the guide RNA, and an expression vector, to a low dose.

### 6. Guide RNA for Editing Target RNA Sequence

The guide RNA for editing a target RNA sequence of the present invention is a guide RNA comprising an antisense nucleotide sequence complementary to a portion of the target RNA sequence, a short-chain ADAR-recruiting nucleotide sequence, and at least one functional nucleotide sequence. Individual sequences that constitute the guide RNA may be directly connected with one another, or may be indirectly connected with one another via linker nucleotide sequences.

In a certain aspect, the short-chain ADAR-recruiting nucleotide sequence and the at least one functional nucleotide sequence are connected with one another via a linker nucleotide sequence(s). In a certain aspect, the at least one functional nucleotide sequence and the antisense nucleotide sequence are connected with one another via a linker nucleotide sequence(s). In a certain aspect, two or more of the functional nucleotide sequences are connected with one another via a linker nucleotide sequence(s). In a certain aspect, the short-chain ADAR-recruiting nucleotide sequence and the at least one functional nucleotide sequence are connected with one another via a linker nucleotide sequence(s); and further, the functional nucleotide sequence and the antisense nucleotide sequence, the linker nucleotide sequence and the functional nucleotide sequence, and/or two or more of the functional nucleotide sequences may each be connected with each other via other short linker nucleotide sequences.

In a certain aspect, the guide RNA for editing a target RNA sequence of the present invention is any of the following guide RNAs:

a guide RNA, in which an antisense nucleotide sequence complementary to a portion of the target RNA sequence, a short-chain ADAR-recruiting nucleotide sequence, and at least one functional nucleotide sequence are connected with one another, in this order, from the 5′-terminal side to the 3′-terminal side,

a guide RNA, in which an antisense nucleotide sequence complementary to a portion of the target RNA sequence, a short-chain ADAR-recruiting nucleotide sequence, and at least one functional nucleotide sequence are connected with one another, in this order, from the 5′-terminal side to the 3′-terminal side, and further, at least one functional nucleotide sequence is further connected with the 5′-terminal side of the antisense nucleotide sequence,

a guide RNA, comprising an antisense nucleotide sequence complementary to a portion of the target RNA sequence, a short-chain ADAR-recruiting nucleotide sequence, a linker nucleotide sequence, and at least one functional nucleotide sequence, a guide RNA, comprising an antisense nucleotide sequence complementary to a portion of the target RNA sequence, a short-chain ADAR-recruiting nucleotide sequence, a linker nucleotide sequence, and at least one functional nucleotide sequence, wherein the at least one functional nucleotide sequence is connected with the short-chain ADAR-recruiting nucleotide sequence via the linker nucleotide sequence,

a guide RNA, comprising an antisense nucleotide sequence complementary to a portion of the target RNA sequence, a short-chain ADAR-recruiting nucleotide sequence, a linker nucleotide sequence, and at least one functional nucleotide sequence, wherein the antisense nucleotide sequence, the short-chain ADAR-recruiting nucleotide sequence, the linker nucleotide sequence, and the at least one functional nucleotide sequence are connected with one another, in this order, from the 5′-terminal side to the 3′-terminal side, or

a guide RNA, comprising an antisense nucleotide sequence complementary to a portion of the target RNA sequence, a short-chain ADAR-recruiting nucleotide sequence, a linker nucleotide sequence, and at least one functional nucleotide sequence, wherein the antisense nucleotide sequence, the short-chain ADAR-recruiting nucleotide sequence, the linker nucleotide sequence, and the at least one functional nucleotide sequence are connected with one another, in this order, from the 5′-terminal side to the 3′-terminal side, and further, at least one functional nucleotide sequence is connected with the 5′-terminal side of the antisense nucleotide sequence.

In a certain aspect, the guide RNA for editing a target RNA sequence of the present invention is any of the following guide RNAs:

a guide RNA, comprising an antisense nucleotide sequence complementary to a portion of the target RNA sequence, a short-chain ADAR-recruiting nucleotide sequence, and at least one nucleotide sequence associated with the intracellular stabilization and/or intracellular localization of the guide RNA,

a guide RNA, comprising an antisense nucleotide sequence complementary to a portion of the target RNA sequence, a short-chain ADAR-recruiting nucleotide sequence, and at least one of a nucleotide sequence that forms a G-quadruplex structure and/or a nucleotide sequence that forms a stem-loop structure,

a guide RNA, comprising an antisense nucleotide sequence complementary to a portion of the target RNA sequence, a short-chain ADAR-recruiting nucleotide sequence, and at least one U6 snRNA sequence,

a guide RNA, comprising an antisense nucleotide sequence complementary to a portion of the target RNA sequence, a short-chain ADAR-recruiting nucleotide sequence, and at least one nucleotide sequence that forms a 1 to 4 layered G-quadruplex structure,

a guide RNA, comprising an antisense nucleotide sequence complementary to a portion of the target RNA sequence, a short-chain ADAR-recruiting nucleotide sequence, and at least one nucleotide sequence that forms a stem-loop structure,

a guide RNA, comprising an antisense nucleotide sequence complementary to a portion of the target RNA sequence, a short-chain ADAR-recruiting nucleotide sequence, and at least one BoxB sequence-derived nucleotide sequence, or

a guide RNA, comprising an antisense nucleotide sequence complementary to a portion of the target RNA sequence, a short-chain ADAR-recruiting nucleotide sequence, and at least one functional nucleotide sequence consisting of the nucleotide sequence as set forth in SEQ ID NO: 6, 7, 8, or 9, or a nucleotide sequence comprising a deletion, substitution, insertion and/or addition of 1 to 3 nucleotides with respect to the nucleotide sequence as set forth in SEQ ID NO: 6, 7, 8, or 9, and having functions.

In a certain aspect, the guide RNA for editing a target RNA sequence of the present invention is any of the following guide RNAs:

a guide RNA, comprising an antisense nucleotide sequence complementary to a portion of the target RNA sequence, a short-chain ADAR-recruiting nucleotide sequence consisting of 14 to 34 nucleotides, and at least one functional nucleotide sequence,

a guide RNA, comprising an antisense nucleotide sequence complementary to a portion of the target RNA sequence, a short-chain ADAR-recruiting nucleotide sequence consisting of 16 nucleotides, and at least one functional nucleotide sequence,

a guide RNA, comprising an antisense nucleotide sequence complementary to a portion of the target RNA sequence, a short-chain ADAR-recruiting nucleotide sequence consisting of 14 to 34 nucleotides, which comprises a loop portion consisting of 4 or 5 nucleotides, and at least one functional nucleotide sequence,

a guide RNA, comprising an antisense nucleotide sequence complementary to a portion of the target RNA sequence, a short-chain ADAR-recruiting nucleotide sequence consisting of 16 nucleotides, which comprises a loop portion consisting of 4 or 5 nucleotides, and at least one functional nucleotide sequence,

a guide RNA, comprising an antisense nucleotide sequence complementary to a portion of the target RNA sequence, a short-chain ADAR-recruiting nucleotide sequence consisting of 14 to 34 nucleotides, wherein a stem portion has a nucleotide that forms a mismatched base pair, and at least one functional nucleotide sequence,

a guide RNA, comprising an antisense nucleotide sequence complementary to a portion of the target RNA sequence, a short-chain ADAR-recruiting nucleotide sequence consisting of 16 nucleotides, wherein a stem portion has a nucleotide that forms a mismatched base pair, and at least one functional nucleotide sequence,

a guide RNA, comprising an antisense nucleotide sequence complementary to a portion of the target RNA sequence, a short-chain ADAR-recruiting nucleotide sequence consisting of 14 to 34 nucleotides, wherein a loop portion consists of 4 or 5 nucleotides and a stem portion has a nucleotide that forms a mismatched base pair, and at least one functional nucleotide sequence, or

a guide RNA, comprising an antisense nucleotide sequence complementary to a portion of the target RNA sequence, a short-chain ADAR-recruiting nucleotide sequence consisting of 16 nucleotides, wherein a loop portion consists of 4 or 5 nucleotides and a stem portion has a nucleotide that forms a mismatched base pair, and at least one functional nucleotide sequence.

In a certain aspect, the guide RNA for editing a target RNA sequence of the present invention is a guide RNA comprising an antisense nucleotide sequence complementary to a portion of the target RNA sequence, a short-chain ADAR-recruiting nucleotide sequence consisting of the nucleotide sequence as set forth in SEQ ID NO: 1, 2, 3, or 4, or a nucleotide sequence comprising a deletion, substitution, insertion and/or addition of 1 to 3 nucleotides with respect to the nucleotide sequence as set forth in SEQ ID NO: 1, 2, 3, or 4, and forming a stem-loop structure, and at least one functional nucleotide sequence.

In a certain aspect, the guide RNA for editing a target RNA sequence of the present invention is a guide RNA comprising an antisense nucleotide sequence complementary to a portion of the target RNA sequence, a short-chain ADAR-recruiting nucleotide sequence, a linker nucleotide sequence consisting of 15 to 25 nucleotides, and at least one functional nucleotide sequence. In a certain aspect, the guide RNA for editing a target RNA sequence of the present invention is a guide RNA comprising an antisense nucleotide sequence complementary to a portion of the target RNA sequence, a short-chain ADAR-recruiting nucleotide sequence, a linker nucleotide sequence consisting of 15 to 25 nucleotides and optionally having a stem-loop structure, and at least one functional nucleotide sequence.

In a certain aspect, the guide RNA for editing a target RNA sequence of the present invention is a guide RNA comprising:

an antisense nucleotide sequence complementary to a portion of the target RNA sequence,

a short-chain ADAR-recruiting nucleotide sequence, consisting of the nucleotide sequence as set forth in SEQ ID NO: 1 (ARR14), SEQ ID NO: 2 (ARR16), SEQ ID NO: 3 (ARR24), or SEQ ID NO: 4 (ARR34), or a nucleotide sequence comprising a deletion, substitution, insertion and/or addition of 1 to 3 nucleotides with respect to the nucleotide sequence as set forth in SEQ ID NO: 1, 2, 3, or 4, and forming a stem-loop structure, and

at least one functional nucleotide sequence, consisting of the nucleotide sequence as set forth in SEQ ID NO: 6 (3Gq), SEQ ID NO: 7 (BoxB), or SEQ ID NO: 8 (U6 upstream) and SEQ ID NO: 9 (U6 downstream), or a nucleotide sequence comprising a deletion, substitution, insertion and/or addition of 1 to 3 nucleotides with respect to any nucleotide sequence as set forth in SEQ ID NO: 6, 7, 8, or 9, and having functions.

In a certain aspect, the guide RNA for editing a target RNA sequence of the present invention is a guide RNA comprising:

an antisense nucleotide sequence complementary to a portion of the target RNA sequence,

a short-chain ADAR-recruiting nucleotide sequence, consisting of the nucleotide sequence as set forth in SEQ ID NO: 1 (ARR14), SEQ ID NO: 2 (ARR16), SEQ ID NO: 3 (ARR24), or SEQ ID NO: 4 (ARR34), or a nucleotide sequence comprising a deletion, substitution, insertion and/or addition of 1 to 3 nucleotides with respect to the nucleotide sequence as set forth in SEQ ID NO: 1, 2, 3, or 4, and forming a stem-loop structure,

a linker nucleotide sequence, consisting of the nucleotide sequence as set forth in SEQ ID NO: 10 (add(20)), or a nucleotide sequence comprising a deletion, substitution, insertion and/or addition of 1 to 3 nucleotides with respect to the nucleotide sequence as set forth in SEQ ID NO: 10, and forming a stem-loop structure, and

at least one functional nucleotide sequence, consisting of the nucleotide sequence as set forth in SEQ ID NO: 6 (3Gq), SEQ ID NO: 7 (BoxB), or SEQ ID NO: 8 (U6 upstream) and SEQ ID NO: 9 (U6 downstream), or a nucleotide sequence comprising a deletion, substitution, insertion and/or addition of 1 to 3 nucleotides with respect to any nucleotide sequence as set forth in SEQ ID NO: 6, 7, 8, or 9, and having functions nucleotide sequence.

In a certain aspect, the guide RNA for editing a target RNA sequence of the present invention is a guide RNA comprising an antisense nucleotide sequence complementary to a portion of the target RNA sequence, a short-chain ADAR-recruiting nucleotide sequence, and at least one functional nucleotide sequence, and the present guide RNA has any of the following characteristics:

(1) the antisense nucleotide sequence complementary to a portion of the target RNA sequence that is connected with the 5′-terminal side of the short-chain ADAR-recruiting nucleotide sequence,

(2) the antisense nucleotide sequence complementary to a portion of the target RNA sequence that is connected with the 5′-terminal side of the short-chain ADAR-recruiting nucleotide sequence, and the functional nucleotide sequence that is connected with the 3′-terminal side of the short-chain ADAR-recruiting nucleotide sequence,

(3) the antisense nucleotide sequence complementary to a portion of the target RNA sequence that is connected with the 5′-terminal side of the short-chain ADAR-recruiting nucleotide sequence, and the functional nucleotide sequence that is connected with the 5′-terminal side of the antisense nucleotide sequence, or

(4) the antisense nucleotide sequence complementary to a portion of the target RNA sequence that is connected with the 5′-terminal side of the short-chain ADAR-recruiting nucleotide sequence, and the functional nucleotide sequence that is connected with both of the 5′-terminal side of the antisense nucleotide sequence and the 3′-terminal side of the short-chain ADAR-recruiting nucleotide sequence.

The guide RNA of the present invention having any of the above-described (1) to (4) may comprise a linker nucleotide sequence. In the guide RNA of the present invention, the antisense nucleotide sequence, the short-chain ADAR-recruiting nucleotide sequence, and the functional nucleotide sequence(s) may be directly connected with one another, or the antisense nucleotide sequence, the short-chain ADAR-recruiting nucleotide sequence, and the functional nucleotide sequence(s) may also be connected with one another via the linker nucleotide sequence. In a certain aspect, the short-chain ADAR-recruiting nucleotide sequence and the functional nucleotide sequence are connected with each other via the linker nucleotide sequence.

In the guide RNA of the present invention having any of the above-described characteristics (1) to (4), the short-chain ADAR-recruiting nucleotide sequence is, in a certain aspect, an ADAR-recruiting nucleotide sequence consisting of 14 to 34 nucleotides, or in a certain aspect, an ADAR-recruiting nucleotide sequence consisting of 16 nucleotides. In a certain aspect, the short-chain ADAR-recruiting nucleotide sequence consists of the nucleotide sequence as set forth in SEQ ID NO: 1, 2, 3, or 4, or a nucleotide sequence comprising a deletion, substitution, insertion and/or addition of 1 to 3 nucleotides with respect to the nucleotide sequence as set forth in SEQ ID NO: 1, 2, 3, or 4, and forming a stem-loop structure.

In a certain aspect, the guide RNA of the present invention having any of the above-described characteristics (1) to (4) comprises at least one functional nucleotide sequence selected from among a Gq sequence, a BoxB sequence-derived sequence, and a U6 snRNA sequence. In a certain aspect, the guide RNA of the present invention having any of the above-described characteristics (1) to (4) comprises, as a functional nucleotide sequence(s), at least one functional nucleotide sequence selected from among: a Gq sequence consisting of the nucleotide sequence as set forth in SEQ ID NO: 6, or a nucleotide sequence comprising a deletion, substitution, insertion and/or addition of 1 to 3 nucleotides with respect to the nucleotide sequence as set forth in SEQ ID NO: 6, and forming a Gq structure; a BoxB sequence-derived sequence consisting of the nucleotide sequence as set forth in SEQ ID NO: 7, or a nucleotide sequence comprising a deletion, substitution, insertion and/or addition of 1 to 3 nucleotides with respect to the nucleotide sequence as set forth in SEQ ID NO: 7, and forming a stem-loop structure; or a U6 snRNA sequence consisting of a combination of the nucleotide sequence as set forth in SEQ ID NO: 8, or a nucleotide sequence comprising a deletion, substitution, insertion and/or addition of 1 to 3 nucleotides with respect to the nucleotide sequence as set forth in SEQ ID NO: 8, and having the function of promoting localization of the guide RNA into the nucleus when the nucleotide sequence is used together with the sequence of the downstream region of the U6 snRNA sequence, and the nucleotide sequence as set forth in SEQ ID NO: 9, or a nucleotide sequence comprising a deletion, substitution, insertion and/or addition of 1 to 3 nucleotides with respect to the nucleotide sequence as set forth in SEQ ID NO: 9, and having the function of promoting localization of the guide RNA into the nucleus when the nucleotide sequence is used together with the sequence of the upstream region of the U6 snRNA sequence.

### <Method for Producing Guide RNA of the Present Invention>

The guide RNA of the present invention can be synthesized based on the sequence information according to a standard polynucleotide synthesis method that is known in the present technical field. In addition, if a certain guide RNA of the present invention is obtained, a mutation is introduced into a predetermined site of the certain guide RNA by applying a method known to those skilled in the art, such as site-directed mutagenesis (Current Protocols in Molecular Biology, 1987, John Wiley & Sons Section), so that another guide RNA of the present invention that maintains the function of inducing ADAR to the target RNA sequence and the function of editing the target RNA sequence can be produced. The guide RNA of the present invention can also be produced using a chemically modified nucleic acid.

<ADAR used in the Present Invention>

The ADAR used in the present invention includes naturally occurring ADAR, and a modified form thereof, as long as it has a deaminase activity. In a certain aspect, the ADAR used in the present invention is eukaryote-derived ADAR, and in another aspect, the present ADAR is mammal-derived ADAR. In a certain aspect, the ADAR used in the present invention is human ADAR, in a certain aspect, the present ADAR is ADAR1 or ADAR2, and in a certain aspect, it is ADAR2. The ADAR1 includes 2 types of splicing variants, ADAR1 p110 and ADAR1 p150. The ADAR used in the present invention is, in a certain aspect, ADAR1 p110 or ADAR1 p150. The ADAR used in the present invention is, in a certain aspect, human ADAR1 or human ADAR2, and it is, in a certain aspect, human ADAR2.

In a certain aspect, the ADAR used in the present invention is a polypeptide comprising a double-stranded RNA binding domain (dsRBD) and having a deaminase enzyme activity (Trends in Biochemical Sciences, 2001, Vol. 26, pp. 376-384, RNA, 2001, Vol. 7, pp. 846-858).

In a certain aspect, the ADAR used in the present invention is a polypeptide that comprises an adenosine deaminase domain and is recruited by the guide RNA of the present invention. In another aspect, the ADAR used in the present invention is a polypeptide that comprises a deaminase domain and can convert the cytidine of the target RNA to uridine.

The ADAR used in the present invention may be a fusion protein with another factor. Examples of a modified form of the ADAR used in the present invention may include a modified form having an adenosine deaminase activity, a modified form having a cytidine deaminase activity, and a modified form having a deaminase activity that recognizes both adenosine and cytidine and converts them to inosine and uridine, respectively. Such a modified form of the ADAR used in the present invention may be a fusion protein with another factor.

In a certain aspect, the ADAR used in the present invention is a polypeptide consisting of the amino acid sequence with Accession No. [NP_001103.1], Accession No. [NP_056648.1] or Accession No. [NP_001102.3], or a polypeptide consisting of an amino acid sequence having an identity of 90% or more to the aforementioned amino acid sequences, or an amino acid sequence comprising a deletion, substitution, insertion and/or addition of 1 to 10 amino acids with respect to the aforementioned amino acid sequences, and having an adenosine deaminase activity. In a certain aspect, the ADAR used in the present invention is a polypeptide consisting of the amino acid sequence as set forth in SEQ ID NO: 12 (human ADAR2), or a polypeptide consisting of an amino acid sequence having an identity of 90% or more to the amino acid sequence as set forth in SEQ ID NO: 12, or an amino acid sequence comprising a deletion, substitution, insertion and/or addition of 1 to 10 amino acids with respect to the amino acid sequence as set forth in SEQ ID NO: 12, and having an adenosine deaminase activity. In a certain aspect, the ADAR used in the present invention may be ADAR that is endogenously present in a eukaryotic cell, or in a certain aspect, the present ADAR may also be ADAR that is exogenously introduced into a eukaryotic cell. With regard to introduction of the ADAR into the eukaryotic cell, the ADAR polypeptide may be directly into the cell, or an expression vector containing a nucleic acid encoding the ADAR may be introduced into the cell.

### <Target RNA Sequence-Editing System of the Present Invention>

The present invention also provides a system for editing a target RNA sequence, comprising the guide RNA of the present invention and ADAR. The system for editing a target RNA sequence, comprising the guide RNA of the present invention and ADAR, includes a kit for editing a target RNA sequence, comprising the guide RNA of the present invention and ADAR, or a method for editing a target RNA sequence, using the guide RNA of the present invention and ADAR. The target RNA sequence-editing system of the present invention is a system comprising: a guide RNA comprising an antisense nucleotide sequence complementary to a portion of the target RNA sequence, a short-chain ADAR-recruiting nucleotide sequence, and at least one functional nucleotide sequence; and ADAR. In a certain aspect, the target RNA sequence-editing system of the present invention is a system comprising: a guide RNA comprising an antisense nucleotide sequence complementary to a portion of the target RNA sequence, a short-chain ADAR-recruiting nucleotide sequence, and at least one functional nucleotide sequence; and human ADAR1 or human ADAR2. The target RNA sequence-editing system of the present invention can be used, either intracellularly or extracellularly. In a certain aspect, the present target RNA sequence-editing system can be used in a eukaryotic cell.

### <Nucleic Acid Encoding Guide RNA of the Present Invention>

The nucleic acid encoding the guide RNA of the present invention is a nucleic acid encoding a guide RNA comprising an antisense nucleotide sequence complementary to a portion of the target RNA sequence, a short-chain ADAR-recruiting nucleotide sequence, and at least one functional nucleotide sequence. Individual sequences comprised in the guide RNA may be directly connected with one another, or may also be connected with one another via a linker nucleotide sequence.

In a certain aspect, the nucleic acid encoding the guide RNA of the present invention is any of the following nucleic acids:

a nucleic acid encoding a guide RNA comprising an antisense nucleotide sequence complementary to a portion of the target RNA sequence, a short-chain ADAR-recruiting nucleotide sequence, and at least one functional nucleotide sequence,

a nucleic acid encoding a guide RNA, in which an antisense nucleotide sequence complementary to a portion of the target RNA sequence, a short-chain ADAR-recruiting nucleotide sequence, and at least one functional nucleotide sequence are connected with one another, in this order, from the 5′-terminal side to the 3′-terminal side,

a nucleic acid encoding a guide RNA, in which an antisense nucleotide sequence complementary to a portion of the target RNA sequence, a short-chain ADAR-recruiting nucleotide sequence, and at least one functional nucleotide sequence are connected with one another, in this order, from the 5′-terminal side to the 3′-terminal side, and further, at least one functional nucleotide sequence is further connected with the 5′-terminal side of the antisense nucleotide sequence,

a nucleic acid encoding a guide RNA comprising an antisense nucleotide sequence complementary to a portion of the target RNA sequence, a short-chain ADAR-recruiting nucleotide sequence, a linker nucleotide sequence, and at least one functional nucleotide sequence,

a nucleic acid encoding a guide RNA comprising an antisense nucleotide sequence complementary to a portion of the target RNA sequence, a short-chain ADAR-recruiting nucleotide sequence, a linker nucleotide sequence, and at least one functional nucleotide sequence, wherein the at least one functional nucleotide sequence is connected with the short-chain ADAR-recruiting nucleotide sequence via the linker nucleotide sequence,

a nucleic acid encoding a guide RNA comprising an antisense nucleotide sequence complementary to a portion of the target RNA sequence, a short-chain ADAR-recruiting nucleotide sequence, a linker nucleotide sequence, and at least one functional nucleotide sequence, wherein the antisense nucleotide sequence, the short-chain ADAR-recruiting nucleotide sequence, the linker nucleotide sequence, and the at least one functional nucleotide sequence are connected with one another, in this order, from the 5′-terminal side to the 3′-terminal side, or

a nucleic acid encoding a guide RNA comprising an antisense nucleotide sequence complementary to a portion of the target RNA sequence, a short-chain ADAR-recruiting nucleotide sequence, a linker nucleotide sequence, and at least one functional nucleotide sequence, wherein the antisense nucleotide sequence, the short-chain ADAR-recruiting nucleotide sequence, the linker nucleotide sequence, and the at least one functional nucleotide sequence are connected with one another, in this order, from the 5′-terminal side to the 3′-terminal side, and further, at least one functional nucleotide sequence is connected with the 5′-terminal side of the antisense nucleotide sequence.

In a certain aspect, the nucleic acid encoding the guide RNA of the present invention is any of the following nucleic acids:

a nucleic acid encoding a guide RNA comprising an antisense nucleotide sequence complementary to a portion of the target RNA sequence, a short-chain ADAR-recruiting nucleotide sequence, and at least one nucleotide sequence associated with the intracellular stabilization and/or intracellular localization of the guide RNA,

a nucleic acid encoding a guide RNA comprising an antisense nucleotide sequence complementary to a portion of the target RNA sequence, a short-chain ADAR-recruiting nucleotide sequence, and at least one of a nucleotide sequence that forms a G-quadruplex structure and/or a nucleotide sequence that forms a stem-loop structure,

a nucleic acid encoding a guide RNA comprising an antisense nucleotide sequence complementary to a portion of the target RNA sequence, a short-chain ADAR-recruiting nucleotide sequence, and at least one U6 snRNA sequence,

a nucleic acid encoding a guide RNA comprising an antisense nucleotide sequence complementary to a portion of the target RNA sequence, a short-chain ADAR-recruiting nucleotide sequence, and at least one nucleotide sequence that forms a 1 to 4 layered G-quadruplex structure,

a nucleic acid encoding a guide RNA comprising an antisense nucleotide sequence complementary to a portion of the target RNA sequence, a short-chain ADAR-recruiting nucleotide sequence, and at least one nucleotide sequence that forms a stem-loop structure, a nucleic acid encoding a guide RNA comprising an antisense nucleotide sequence complementary to a portion of the target RNA sequence, a short-chain ADAR-recruiting nucleotide sequence, and at least one BoxB sequence-derived nucleotide sequence, or

a nucleic acid encoding a guide RNA comprising an antisense nucleotide sequence complementary to a portion of the target RNA sequence, a short-chain ADAR-recruiting nucleotide sequence, and at least one functional nucleotide sequence consisting of the nucleotide sequence as set forth in SEQ ID NO: 6, 7, 8, or 9, or a nucleotide sequence comprising a deletion, substitution, insertion and/or addition of 1 to 3 nucleotides with respect to the nucleotide sequence as set forth in SEQ ID NO: 6, 7, 8, or 9, and having functions.

In a certain aspect, the nucleic acid encoding the guide RNA of the present invention is a nucleic acid encoding any of the following guide RNAs:

a nucleic acid encoding a guide RNA comprising an antisense nucleotide sequence complementary to a portion of the target RNA sequence, a short-chain ADAR-recruiting nucleotide sequence consisting of 14 to 34 nucleotides, and at least one functional nucleotide sequence,

a nucleic acid encoding a guide RNA comprising an antisense nucleotide sequence complementary to a portion of the target RNA sequence, a short-chain ADAR-recruiting nucleotide sequence consisting of 16 nucleotides, and at least one functional nucleotide sequence,

a nucleic acid encoding a guide RNA comprising an antisense nucleotide sequence complementary to a portion of the target RNA sequence, a short-chain ADAR-recruiting nucleotide sequence consisting of 14 to 34 nucleotides, which comprises a loop portion consisting of 4 or 5 nucleotides, and at least one functional nucleotide sequence,

a nucleic acid encoding a guide RNA comprising an antisense nucleotide sequence complementary to a portion of the target RNA sequence, a short-chain ADAR-recruiting nucleotide sequence consisting of 16 nucleotides, which comprises a loop portion consisting of 4 or 5 nucleotides, and at least one functional nucleotide sequence,

a nucleic acid encoding a guide RNA comprising an antisense nucleotide sequence complementary to a portion of the target RNA sequence, a short-chain ADAR-recruiting nucleotide sequence consisting of 14 to 34 nucleotides, wherein a stem portion has a nucleotide that forms a mismatched base pair, and at least one functional nucleotide sequence,

a nucleic acid encoding a guide RNA comprising an antisense nucleotide sequence complementary to a portion of the target RNA sequence, a short-chain ADAR-recruiting nucleotide sequence consisting of 16 nucleotides, wherein a stem portion has a nucleotide that forms a mismatched base pair, and at least one functional nucleotide sequence,

a nucleic acid encoding a guide RNA comprising an antisense nucleotide sequence complementary to a portion of the target RNA sequence, a short-chain ADAR-recruiting nucleotide sequence consisting of 14 to 34 nucleotides, wherein a loop portion consists of 4 or 5 nucleotides and a stem portion has a nucleotide that forms a mismatched base pair, and at least one functional nucleotide sequence, or

a nucleic acid encoding a guide RNA comprising an antisense nucleotide sequence complementary to a portion of the target RNA sequence, a short-chain ADAR-recruiting nucleotide sequence consisting of 16 nucleotides, wherein a loop portion consists of 4 or 5 nucleotides and a stem portion has a nucleotide that forms a mismatched base pair, and at least one functional nucleotide sequence.

In a certain aspect, the nucleic acid encoding the guide RNA of the present invention is a nucleic acid encoding a guide RNA comprising an antisense nucleotide sequence complementary to a portion of the target RNA sequence, a short-chain ADAR-recruiting nucleotide sequence consisting of the nucleotide sequence as set forth in SEQ ID NO: 1, 2, 3, or 4, or a nucleotide sequence comprising a deletion, substitution, insertion and/or addition of 1 to 3 nucleotides with respect to the nucleotide sequence as set forth in SEQ ID NO: 1, 2, 3, or 4, and forming a stem-loop structure, and at least one functional nucleotide sequence.

In a certain aspect, the nucleic acid encoding the guide RNA of the present invention is a nucleic acid encoding a guide RNA comprising an antisense nucleotide sequence complementary to a portion of the target RNA sequence, a short-chain ADAR-recruiting nucleotide sequence, a linker nucleotide sequence consisting of 15 to 25 nucleotides and optionally having a stem-loop structure, and at least one functional nucleotide sequence.

In a certain aspect, the nucleic acid encoding the guide RNA of the present invention is a nucleic acid encoding a guide RNA comprising an antisense nucleotide sequence complementary to a portion of the target RNA sequence, a short-chain ADAR-recruiting nucleotide sequence consisting of the nucleotide sequence as set forth in SEQ ID NO: 1, 2, 3, or 4, or a nucleotide sequence comprising a deletion, substitution, insertion and/or addition of 1 to 3 nucleotides with respect to the nucleotide sequence as set forth in SEQ ID NO: 1, 2, 3, or 4, and forming a stem-loop structure, a linker nucleotide sequence that is the nucleotide sequence as set forth in SEQ ID NO: 10, or a nucleotide sequence comprising a deletion, substitution, insertion and/or addition of 1 to 3 nucleotides with respect to the nucleotide sequence as set forth in SEQ ID NO: 10, and at least one functional nucleotide sequence consisting of the nucleotide sequence as set forth in SEQ ID NO: 6, 7, 8, or 9, or a nucleotide sequence comprising a deletion, substitution, insertion and/or addition of 1 to 3 nucleotides with respect to the nucleotide sequence as set forth in SEQ ID NO: 6, 7, 8, or 9, and having functions.

In a certain aspect, the nucleic acid encoding the guide RNA of the present invention includes a nucleic acid encoding a guide RNA comprising an antisense nucleotide sequence complementary to a portion of the target RNA sequence, a short-chain ADAR-recruiting nucleotide sequence, a linker nucleotide sequence, and at least one functional nucleotide sequence, wherein nucleic acids encoding the short-chain ADAR-recruiting nucleotide sequence, the linker nucleotide sequence, and the at least one functional nucleotide sequence each consist of the following nucleotide sequences, or nucleotide sequences each comprising a deletion, substitution, insertion and/or addition of 1 to 3 nucleotides with respect to the following nucleotide sequences.

The nucleic acid sequence encoding the short-chain ADAR-recruiting nucleotide sequence:
the nucleic acid encoding ARR(16): SEQ ID NO: 13

The nucleic acid sequence encoding the linker nucleotide sequence:
the nucleic acid encoding add(20): SEQ ID NO: 14

The nucleic acids encoding the functional nucleotide sequences:
the nucleic acid encoding 3Gq: SEQ ID NO: 15
the nucleic acid encoding BoxB: SEQ ID NO: 16
the nucleic acid encoding U6 upstream: SEQ ID NO: 17
the nucleic acid encoding U6 downstream: SEQ ID NO: 18

The nucleic acid encoding the guide RNA of the present invention is, for example, a DNA or a modified DNA, and in a certain aspect, the nucleic acid encoding the guide RNA of the present invention is a DNA.

In a certain aspect, the nucleic acid encoding the guide RNA of the present invention is a nucleic acid that is incorporated into an expression vector. In a certain aspect, the nucleic acid encoding the guide RNA of the present invention is a nucleic acid that is incorporated into a plasmid vector. In a certain aspect, the nucleic acid encoding the guide RNA of the present invention is a nucleic acid that is incorporated into a viral vector.

<Nucleic Acid Encoding ADAR used in the Present Invention>

The nucleic acid encoding the ADAR used in the present invention is a nucleic acid encoding the ADAR described in the above <ADAR used in the present invention>. The nucleic acid encoding the ADAR used in the present invention is, in a certain aspect, a nucleic acid consisting of the nucleotide sequence encoding the amino acid sequence with Accession No. [NP_001103.1], Accession No. [NP_056648.1] or Accession No. [NP_001102.3], or a nucleic acid consisting of a nucleotide sequence encoding a polypeptide that consists of an amino acid sequence having an identity of 90% or more to at least one of these sequences or an amino acid sequence comprising a deletion, substitution, insertion and/or addition of 1 to 10 amino acids with respect to at least one of these sequences, and has an adenosine deaminase activity. The nucleic acid encoding the ADAR used in the present invention is, in a certain aspect, a nucleic acid consisting of the nucleotide sequence as set forth in SEQ ID NO: 11, or a nucleic acid encoding a polypeptide that consists of a nucleotide sequence having an identity of 90% or more to the nucleotide sequence as set forth in SEQ ID NO: 11, or a nucleotide sequence comprising a deletion, substitution, insertion and/or addition of 1 to 10 nucleotides with respect to the nucleotide sequence as set forth in SEQ ID NO: 11, and has an adenosine deaminase activity.

### <Expression Vector of the Present Invention>

The present invention also provides an expression vector containing the nucleic acid encoding the guide RNA of the present invention (which is also referred to as “the expression vector of the present invention”). The expression vector of the present invention may further comprise a nucleic acid encoding ADAR. The nucleic acid encoding the guide RNA of the present invention and the nucleic acid encoding the ADAR used in the present invention may be incorporated into a single expression vector, or may also be incorporated into different expression vectors. In a certain aspect, the expression vector of the present invention is a combination of an expression vector containing the nucleic acid encoding the guide RNA of the present invention and an expression vector containing the nucleic acid encoding ADAR. In a certain aspect, the expression vector of the present invention is an expression vector containing the nucleic acid encoding the guide RNA of the present invention and the nucleic acid encoding ADAR.

1. Expression Vector used in the Present Invention

The expression vector used in the present invention is not particularly limited, as long as it can express the guide RNA of the present invention from the nucleic acid encoding the guide RNA of the present invention, and/or can express the ADAR used in the present invention. In a certain aspect, the expression vector used in the present invention is an expression vector that can be used to express the guide RNA of the present invention and/or the ADAR used in the present invention in a human cell. In a certain aspect, examples of the expression vector used in the present invention may include plasmid vectors and viral vectors (e.g. an adenoviral vector, a retroviral vector, and an adeno-associated viral vector).

### 2. Promoter

The expression vector of the present invention may comprise a promoter that is operably linked to the nucleic acid encoding the guide RNA of the present invention and/or the nucleic acid encoding the ADAR used in the present invention. In the present description, the phrase “be operably linked” means that at least one promoter is linked to a nucleic acid, such that a polypeptide or an RNA encoded by the nucleic acid can be expressed in a host cell.

The promoter comprised in the expression vector of the present invention is not particularly limited. A promoter corresponding to RNA polymerase (for example, polII or polIII) suitable for the expression of the nucleic acid encoding the guide RNA of the present invention or the ADAR used in the present invention can be used. In order to allow the guide RNA of the present invention to express, in a certain aspect, a promoter corresponding to polII or polIII can be used, and in another aspect, a promoter corresponding to polIII can be used. In order to allow the ADAR used in the present invention to express, in a certain aspect, a promoter corresponding to polII can be used.

Examples of the promoter corresponding to polII may include a CMV (cytomegalovirus)-derived promoter, an SV40 (simian virus 40) promoter, an RSV (respiratory syncytial virus) promoter, an EF1α (Elongation factor 1α) promoter, and a CAG promoter. Examples of the promoter corresponding to polIII may include a human U6 small nuclear RNA (snRNA) promoter (U6) (Nat. Biotechnol., 2002, Vol. 20, pp. 497-500), a highly sensitive U6 promoter (Nucleic Acids Research, 2003, Vol. 31, p.e100), a human H1 promoter, and other viral and eukaryotic cell promoters known to those skilled in the art, but the examples are not limited thereto.

The expression vector of the present invention may further comprise a translation initiation codon, a translation termination codon, a purine base (G or A) preferable for the transcription start point of polIII, a polyA signal, a terminator sequence made of consecutive T residues for polIII, an enhancer, an untranslated region, a splicing junction, etc., depending on the type of a promoter or host cells used, etc.

### 3. Expression Vector of the Present Invention

The expression vector of the present invention is an expression vector containing the nucleic acid encoding the guide RNA of the present invention. In a certain aspect, the expression vector of the present invention is a combination of an expression vector containing the nucleic acid encoding the guide RNA of the present invention and an expression vector containing the nucleic acid encoding ADAR. In a certain aspect, the expression vector of the present invention is an expression vector containing the nucleic acid encoding the guide RNA of the present invention and the nucleic acid encoding ADAR.

The expression vector of the present invention is an expression vector containing a nucleic acid encoding a guide RNA, wherein the guide RNA comprises an antisense nucleotide sequence complementary to a portion of the target RNA sequence, a short-chain ADAR-recruiting nucleotide sequence, and at least one functional nucleotide sequence.

In a certain aspect, the expression vector of the present invention is an expression vector containing a nucleic acid encoding a guide RNA, wherein the guide RNA comprises an antisense nucleotide sequence complementary to a portion of the target RNA sequence, a short-chain ADAR-recruiting nucleotide sequence, a linker nucleotide sequence, and at least one functional nucleotide sequence.

### <Host Cell of the Present Invention>

The present invention also provides a host cell, into which the nucleic acid encoding the guide RNA of the present invention is introduced (which is also referred to as “the host cell of the present invention”). In a certain aspect, the host cell of the present invention is a host cell, into which an expression vector containing the nucleic acid encoding the guide RNA of the present invention is introduced. In a certain aspect, the host cell of the present invention is a host cell, into which an expression vector containing the nucleic acid encoding the guide RNA of the present invention and the nucleic acid encoding ADAR is introduced. In a certain aspect, the host cell of the present invention is a host cell, into which an expression vector containing the nucleic acid encoding the guide RNA of the present invention and an expression vector containing the nucleic acid encoding ADAR are introduced. In a certain aspect, the host cell of the present invention is a host cell, into which the expression vector of the present invention that is a plasmid vector is introduced. In a certain aspect, the host cell of the present invention is a host cell, into which a plasmid vector for producing the expression vector of the present invention that is a viral vector is introduced.

The host cell, into which the expression vector of the present invention is introduced, is not particularly limited. A cell known in the present technical field can be selected, as long as it is a cell that can be used in replication of the nucleic acid encoding the guide RNA of the present invention. Examples of the host cell that can be used in replication of the vector may include various cells, such as natural cells commonly used in the technical field of the present invention or artificially established cells. Specific examples of the host cell that can be used in replication of the vector may include animal cells (e.g. CHO cells, HEK293 cells, etc.), insect cells (e.g. Sf9 cells, etc.), bacteria (Escherichia coli, etc.), and yeasts (genus Saccharomyces, genus Pichia, etc.). In a certain aspect, Escherichia coli can be used as a host cell. Introduction itself can be carried out according to a known method (Green, M. R. and Sambrook, J., Molecular Cloning: A Laboratory Manual, 4th Edition, Cold Spring Harbor Laboratory Press, 2012).

### <Method for Producing Nucleic Acid Encoding Guide RNA of the Present Invention and Expression Vector of the Present Invention>

The nucleic acid encoding the guide RNA of the present invention (hereinafter also referred to as “the nucleic acid of the present invention”) can be synthesized based on the sequence described in the present description or publicly available sequence information, according to a standard polynucleotide synthesis method that is known in the present technical field. In addition, if a certain nucleic acid of the present invention is obtained, a mutation is introduced into a predetermined site of the certain nucleic acid by applying a method known to those skilled in the art, such as site-directed mutagenesis (Current Protocols in Molecular Biology, 1987, John Wiley & Sons Section), so that another nucleic acid of the present invention can be produced.

The present invention includes a method for producing a nucleic acid or an expression vector, comprising a step of culturing host cells, into which the nucleic acid of the present invention or an expression vector containing the nucleic acid of the present invention is introduced. In a certain aspect, the method for producing the nucleic acid of the present invention comprises a step of culturing host cells, into which the nucleic acid of the present invention is introduced, so that the nucleic acid of the present invention is replicated. In a certain aspect, the method for producing the nucleic acid and expression vector of the present invention comprises a step of culturing host cells, into which an expression vector containing the nucleic acid of the present invention is introduced, so that the expression vector of the present invention is replicated. When the expression vector of the present invention is a viral vector, the method for producing the expression vector of the present invention comprises a step of culturing host cells, into which a plasmid vector used in generation of a viral vector containing the nucleic acid of the present invention is introduced, or host cells, into which a viral vector containing the nucleic acid of the present invention is introduced, and then purifying a viral vector produced in the host cells. Such a viral vector can be produced according to a method known to those skilled in the art.

The method for producing the nucleic acid of the present invention and the expression vector of the present invention may include a step of recovering a culture solution of the host cells to obtain a lysate (lysis solution). Such a lysate can be obtained, for example, by treating the recovered culture solution according to an alkaline lysis method or a boiling method. The method for producing the nucleic acid and expression vector of the present invention may further comprise a step of purifying a nucleic acid or an expression vector from the lysate. For purification of a nucleic acid or an expression vector from the lysate, ion exchange chromatography and/or hydrophobic interaction chromatography can be used.

When the expression vector of the present invention is a viral vector, in order to purify a viral vector from the lysate, cesium chloride density-gradient centrifugation, sucrose gradient centrifugation, iodixanol density-gradient centrifugation, ultrafiltration, diafiltration, affinity chromatography, ion exchange chromatography, polyethylene glycol precipitation, ammonium sulfate precipitation, or the like can be used.

### <Pharmaceutical Composition of the Present Invention>

The present invention also provides a pharmaceutical composition comprising the guide RNA of the present invention, the nucleic acid of the present invention, or the expression vector of the present invention, and a pharmaceutically acceptable excipient (also referred to as “the pharmaceutical composition of the present invention”).

In a certain aspect, the pharmaceutical composition of the present invention is a pharmaceutical composition comprising the guide RNA of the present invention and a pharmaceutically acceptable excipient. In a certain aspect, the pharmaceutical composition of the present invention is a pharmaceutical composition comprising the guide RNA of the present invention, ADAR, and a pharmaceutically acceptable excipient.

In a certain aspect, the pharmaceutical composition of the present invention is a pharmaceutical composition comprising the nucleic acid of the present invention and a pharmaceutically acceptable excipient. In a certain aspect, the pharmaceutical composition of the present invention is a pharmaceutical composition comprising the nucleic acid of the present invention, ADAR, and a pharmaceutically acceptable excipient.

In a certain aspect, the pharmaceutical composition of the present invention is a pharmaceutical composition comprising the expression vector of the present invention and a pharmaceutically acceptable excipient. In a certain aspect, the pharmaceutical composition of the present invention is a pharmaceutical composition comprising an expression vector containing the nucleic acid encoding the guide RNA of the present invention and the nucleic acid encoding ADAR, and a pharmaceutically acceptable excipient. In a certain aspect, the pharmaceutical composition of the present invention is a pharmaceutical composition comprising an expression vector containing the nucleic acid encoding the guide RNA of the present invention, a vector containing the nucleic acid encoding ADAR, and a pharmaceutically acceptable excipient.

In a certain aspect, the pharmaceutical composition of the present invention is a pharmaceutical composition comprising an expression vector containing the nucleic acid encoding the guide RNA of the present invention and the nucleic acid encoding human ADAR1 or human ADAR2, and a pharmaceutically acceptable excipient. In a certain aspect, the pharmaceutical composition of the present invention is a pharmaceutical composition comprising an expression vector containing the nucleic acid encoding the guide RNA of the present invention, a vector containing the nucleic acid encoding human ADAR1 or human ADAR2, and a pharmaceutically acceptable excipient. In a certain aspect, the pharmaceutical composition of the present invention is a pharmaceutical composition comprising an expression vector containing the nucleic acid encoding the guide RNA of the present invention and the nucleic acid encoding ADAR, wherein the nucleic acid consists of a nucleotide sequence encoding the amino acid sequence as set forth in SEQ ID NO: 12 or a nucleotide sequence encoding a polypeptide that consists of an amino acid sequence having an identity of 90% or more to the amino acid sequence as set forth in SEQ ID NO: 12 and has an adenosine deaminase activity, and a pharmaceutically acceptable excipient. In a certain aspect, the pharmaceutical composition of the present invention is a pharmaceutical composition comprising: an expression vector containing the nucleic acid encoding the guide RNA of the present invention; an expression vector containing the nucleic acid encoding ADAR, wherein the nucleic acid consists of a nucleotide sequence encoding the amino acid sequence as set forth in SEQ ID NO: 12 or a nucleotide sequence encoding a polypeptide that consists of an amino acid sequence having an identity of 90% or more to the amino acid sequence as set forth in SEQ ID NO: 12 and has an adenosine deaminase activity; and a pharmaceutically acceptable excipient.

The pharmaceutical composition of the present invention can be prepared using excipients commonly used in the present technical field, such as pharmaceutical excipients or pharmaceutical carriers, according to a commonly used method. Examples of the dosage form of such a pharmaceutical composition may include parenteral agents such as injections and intravenous drips, which can be administered via intravenous administration, subcutaneous administration, intracutaneous administration, intramuscular administration, etc. For formulation of the present pharmaceutical composition, excipients, carriers, additives and the like, which depend on the aforementioned dosage forms, can be used within a pharmaceutically acceptable range.

The applied dose of the guide RNA of the present invention, the nucleic acid of the present invention, an expression vector containing the nucleic acid encoding the guide RNA of the present invention, or an expression vector containing the nucleic acid encoding the guide RNA of the present invention and the nucleic acid encoding ADAR, is different depending on the degree of symptoms or age of a patient, the dosage form of a preparation used, etc. For example, it can be used at a dose of approximately 0.001 mg/kg to 100 mg/kg. Moreover, the guide RNA of the present invention, the nucleic acid of the present invention, an expression vector containing the nucleic acid encoding the guide RNA of the present invention, or an expression vector containing the nucleic acid encoding the guide RNA of the present invention and the nucleic acid encoding ADAR is added in an amount corresponding to the aforementioned applied dose, so as to formulation a preparation.

The applied dose of the expression vector containing the ADAR used in the present invention or the nucleic acid encoding the ADAR can be appropriately adjusted, depending on cells in which the target RNA is present, etc. The expression vector can be used, for example, at a dose of approximately 0.001 mg/kg to 100 mg/kg.

In a certain aspect, the disease, which can be prevented or treated with the guide RNA, the nucleic acid, or the expression vector of the present invention, is a hereditary disease, and in a certain aspect, it is any given disease, to which a modification of one or more adenosine or cytidine residues in the target RNA provides an advantageous change. The pharmaceutical composition of the present invention can be used as a preventive or therapeutic agent for any given disease, to which a modification of one or more adenosine or cytidine residues in the target RNA provides an advantageous change. In addition, the pharmaceutical composition of the present invention can be used as a preventive or therapeutic agent for any given disease, to which a modification of one or more adenosine residues in the target RNA provides an advantageous change.

The present invention includes a pharmaceutical composition for use in preventing or treating any given disease, to which a modification of one or more adenosine or cytidine residues in the target RNA provides an advantageous change, wherein the pharmaceutical composition comprises the guide RNA of the present invention, the nucleic acid of the present invention, or the expression vector of the present invention. In addition, the present invention includes a method for preventing or treating any given disease, to which a modification of one or more adenosine or cytidine residues in the target RNA provides an advantageous change, wherein the method comprises a step of administering to a patient, a preventively effective amount or therapeutically effective amount of the guide RNA of the present invention, the nucleic acid of the present invention, or the expression vector of the present invention. Moreover, the present invention includes the guide RNA of the present invention, the nucleic acid of the present invention, or the expression vector of the present invention, which is for use in preventing or treating any given disease, to which a modification of one or more adenosine or cytidine residues in the target RNA provides an advantageous change. Furthermore, the present invention includes use of the guide RNA of the present invention, the nucleic acid of the present invention, or the expression vector of the present invention, in the production of a pharmaceutical composition for preventing or treating any given disease, to which a modification of one or more adenosine or cytidine residues in the target RNA provides an advantageous change.

<Method for Editing Target RNA Sequence, using Guide RNA of the Present Invention>

The present invention also provides a method for editing a target RNA sequence, using the guide RNA of the present invention (also referred to as “the editing method of the present invention”). In a certain aspect, the editing method of the present invention includes a method by which a target RNA sequence is allowed to react with the guide RNA of the present invention to form a complex, and ADAR recruited by the guide RNA converts the adenosine of the target RNA sequence to inosine. Herein, the adenosine on the target RNA sequence that is to be converted to inosine may form a mismatched base pair with an antisense nucleotide sequence. In a certain aspect, the editing method of the present invention includes a method by which a target RNA sequence is allowed to react with the guide RNA of the present invention to form a complex, and ADAR recruited by the guide RNA converts the cytidine of the target RNA sequence to uridine. Herein, the cytidine on the target RNA sequence that is to be converted to uridine may form a mismatched base pair with an antisense nucleotide sequence.

The editing method comprises: (i) a step of introducing a guide RNA comprising an antisense nucleotide sequence complementary to a portion of the target RNA sequence, a short-chain ADAR-recruiting nucleotide sequence, and at least one functional nucleotide sequence, or a nucleic acid encoding the guide RNA, into cells, (ii) a step of recruiting ADAR by the guide RNA, (iii) a step of forming a complex of the target RNA sequence and the guide RNA; (iv) a step of converting the adenosine of the target RNA sequence to inosine by the ADAR recruited by the guide RNA. The editing method may comprise a step of introducing ADAR or a nucleic acid encoding the ADAR, and a guide RNA or a nucleic acid encoding the guide RNA, into cells, simultaneously or separately.

The editing method includes a method of converting the adenosine of a target sequence to inosine, or a method of converting the cytidine of a target sequence to uridine, in cells, or in eukaryotic cells according to a certain aspect, or in mammalian cells according to a certain aspect. Further, when such a target RNA sequence is present in a coding region, the editing method may comprise a step of reading inosine as guanosine upon translation.

### EXAMPLES

The steps described in the following Examples can be carried out according to known methods, unless otherwise particularly specified. In addition, in steps in which commercially available kits or reagents, etc. were used, experiments were carried out according to protocols included therewith, unless otherwise particularly specified.

### Example 1

### Production of Guide RNA Expression Plasmid Comprising Gq Sequence and/or BoxB Sequence-Derived Sequence as Functional Nucleotide Sequence(s)

A guide RNA expression plasmid comprising a Gq sequence and/or a BoxB sequence-derived sequence as functional nucleotide sequence(s) and also comprising ARR(16) as an ADAR-recruiting nucleotide sequence was produced as follows. Specifically, DNA sequences encoding 6 types of guide RNAs (please refer to the below-mentioned <Names of guide RNAs and sequence numbers of DNAs encoding guide RNAs>) were each inserted into a site located downstream of an H1 RNA polymerase III promoter (hereinafter referred to as “H1 promoter”) of a pSUPER.neo vector (Oligoengine, Catalog No. VEC-PBS-0004), using the restriction enzymes BglII (on the 5′-terminal side) and HindIII (on the 3′-terminal side). Upon production of the DNA sequences to be inserted, the DNA sequences were produced according to either DNA synthesis (FASMAC, Thermo Fisher Scientific), or a method of annealing a forward oligo and a reverse oligo (as described below) that were synthesized to form restriction enzyme ends.
Forward oligo: 5′-GATC-“DNA sequence encoding guide RNA excluding restriction enzyme recognition sequence”-3′
Reverse oligo: 5′-AGCT- “reverse complementary sequence of DNA sequence encoding guide RNA excluding restriction enzyme recognition sequence”-3′

The produced guide RNA expression plasmids are collectively referred to as “pSUPERneo_H1ADg.”

<Names of guide RNAs and Sequence Numbers of DNAs Encoding Guide RNAs>

ASR(24)-ARR(16)add-3Gq: SEQ ID NO: 19

ASR(24)-ARR(16)add-BoxB: SEQ ID NO: 20

BoxB-ASR(24)-ARR(16)add-3Gq: SEQ ID NO: 21

ASR(24)-ARR(16)-3Gq: SEQ ID NO: 22

ASR(24)-ARR(16)-BoxB: SEQ ID NO: 23

BoxB-ASR(24)-ARR(16)-3Gq: SEQ ID NO: 24

The DNA sequence comprises a purine base (G or A) preferable for the transcription start point of polIII on the 5′-terminal side, and a terminator sequence of polIII on the 3′-terminal side. Moreover, the DNA sequence comprises the restriction enzyme BglII recognition sequence (AGATCT) and the restriction enzyme HindII recognition sequence (AAGCTT), which are used in vector cloning, at the 5′-terminus and at the 3′-terminus, respectively.

Herein, ASR(24) means an antisense nucleotide sequence consisting of 24 nucleotides complementary to a part of a reporter mRNA (Rluc-W104X) for detection of RNA editing used in Example 3, and the ASR(24) consists of an RNA sequence encoded by a DNA sequence from nucleotide numbers 10 to 33 as set forth in SEQ ID NO: 19. ARR(16) consists of the RNA sequence as set forth in SEQ ID NO: 2, and the nucleic acid encoding ARR(16) consists of the DNA sequence as set forth in SEQ ID NO: 13. Moreover, “add” consists of the RNA sequence as set forth in SEQ ID NO: 10, and the nucleic acid encoding the “add” consists of the DNA sequence as set forth in SEQ ID NO: 14. Furthermore, 3Gq consists of the RNA sequence as set forth in SEQ ID NO: 6, and the nucleic acid encoding the 3Gq consists of the DNA sequence as set forth in SEQ ID NO: 15. Further, BoxB consists of the RNA sequence as set forth in SEQ ID NO: 7, and the nucleic acid encoding the BoxB consists of the DNA sequence as set forth in SEQ ID NO: 16.

An outline of the DNA sequences encoding individual guide RNAs is shown in Table 1. In Table 1, the term “5′-” means the functional nucleotide sequence on the 5′-terminal side, and the “3′-” means the functional nucleotide sequence on the 3′-terminal side. The term “SEQ ID NO:” means the DNA sequence number encoding each guide RNA.

E. coli DH5α Competent Cells (Takara Bio, Inc., Catalog No. 9057; hereinafter referred to as an “Escherichia coli strain DH5α”) or OneShot Stbl3 Chemically Competent E. coli (Thermo Fisher Scientific, Catalog No. C737303; hereinafter referred to as an “Escherichia coli strain Stbl3”) were transformed with the plasmid pSUPERneo_H1ADg, and were then subjected to a liquid culture. The culture solution was centrifuged, and the E. coli was then collected. Thereafter, plasmid extraction and purification were carried out using NucleoBond(registered trademark) Xtra Midi Plus EF (Takara Bio, Inc., Catalog No. U0422B), so as to obtain the amplified plasmid pSUPERneo_H1ADg.

Besides, the guide RNA expression plasmids used as controls or comparative examples were produced in the same manner as that described above, using the DNA sequences (as shown below) encoding the guide RNAs.

### <Name of Guide RNA and Sequence Number of DNA Encoding Guide RNA (Control)>

ASR(24)-ARR(40): SEQ ID NO: 27

### <Names of Guide RNAs and Sequence Numbers of DNAs Encoding Guide RNAs (Comparative Examples)>

ASR(24)-ARR(16)add: SEQ ID NO: 28

ASR(24)-ARR(16): SEQ ID NO: 29

ASR(24)-ARR(40)add: SEQ ID NO: 30

ASR(24)-ARR(40)add-3Gq: SEQ ID NO: 31

ASR(24)-ARR(40)add-BoxB: SEQ ID NO: 32

BoxB-ASR(24)-ARR(40)add-3Gq: SEQ ID NO: 33

ASR(24)-ARR(40)-3Gq: SEQ ID NO: 35

ASR(24)-ARR(40)-BoxB: SEQ ID NO: 36

BoxB-ASR(24)-ARR(40)-3Gq: SEQ ID NO: 37

The DNA sequence comprises a purine base (G or A) preferable for the transcription start point of polIII on the 5′-terminal side, and a terminator sequence of polIII on the 3′-terminal side. Moreover, the DNA sequence comprises the restriction enzyme BglII recognition sequence (AGATCT) and the restriction enzyme HindII recognition sequence (AAGCTT), which are used in vector cloning, at the 5′-terminus and at the 3′-terminus, respectively.

An outline of the DNA sequences encoding individual guide RNAs is shown in Table 1.

### Example 2

### Production of Guide RNA Expression Plasmid, to which U6 snRNA Sequence is Added

A vector was constructed by substituting the H1 promoter sequence of a pSUPER.neo vector with a human U6 RNA polymerase III (hU6) promoter sequence (SEQ ID NO: 39) (hereinafter referred to as a “pSUPER.neo-U6 vector”). A fragment comprising a hU6 promoter sequence was amplified by using, as a template, pBAsi-hU6 Neo DNA (Takara Bio, Inc., Catalog No. 3227) having a hU6 promoter, and also using primers to which individual restriction enzyme sites were added (i.e. EcoRI on the 5′-terminal side and BglII on the 3′-terminal side), according to a standard PCR method. For the reaction, Tks Gflex DNA polymerase (Takara Bio, Inc., Catalog No. R060A) was used. The obtained hU6 promoter fragment was inserted into the pSUPER.neo vector, using the restriction enzymes EcoRI (on the 5′-terminal side) and BglII (on the 3′-terminal side). The constructed pSUPER.neo-U6 vector was amplified using the Escherichia coli strain DH5α or the Escherichia coli strain Stbl3.

A guide RNA expression plasmid, to which a U6 snRNA sequence was added, was constructed as follows. DNA sequences encoding two types of guide RNAs (as shown below) were inserted into a site located downstream of the hU6 promoter of the pSUPER.neo-U6 vector, using the restriction enzymes BglII (on the 5′-terminal side) and HindIII (on the 3′-terminal side). The DNA sequences to be inserted were produced by the same method as that of Example 1.

### <Names of Guide RNAs and Sequence Numbers of DNAs Encoding Guide RNAs>

U6 upstream-ASR(24)-ARR(16)add-U6 downstream: SEQ ID NO: 25

U6 upstream-ASR(24)-ARR(16)-U6 downstream: SEQ ID NO: 26

The DNA sequence comprises a purine base (G or A) preferable for the transcription start point of polIII on the 5′-terminal side, and a terminator sequence of polIII on the 3′-terminal side. Moreover, the DNA sequence comprises the restriction enzyme BglII recognition sequence (AGATCT) and the restriction enzyme HindII recognition sequence (AAGCTT), which are used in vector cloning, at the 5′-terminus and at the 3′-terminus, respectively.

U6 upstream consists of the RNA sequence as set forth in SEQ ID NO: 8, and the nucleic acid encoding the U6 upstream consists of the DNA sequence as set forth in SEQ ID NO: 17. U6 downstream consists of the RNA sequence as set forth in SEQ ID NO: 9, and the nucleic acid encoding the U6 downstream consists of the DNA sequence as set forth in SEQ ID NO: 18.

The obtained plasmids are collectively referred to as “pSUPERneo_U6ADg.” The constructed pSUPERneo_U6ADg plasmid was amplified using the Escherichia coli strain DHSα or the Escherichia coli strain Stbl3 by the same method as that of Example 1.

An outline of the DNA sequences encoding individual guide RNAs is shown in Table 1.

Besides, guide RNA expression plasmids used as comparative examples were produced in the same manner as that described above, using DNA sequences (as shown below) encoding the guideRNAs.

<Names of Guide RNA Sequences and Sequence Numbers of DNAs Encoding those Guide RNA Sequences (Comparative Examples)>

U6 upstream-ASR(24)-ARR(40)add-U6 downstream: SEQ ID NO: 34

U6 upstream-ASR(24)-ARR(40)-U6 downstream: SEQ ID NO: 38

The DNA sequence comprises a purine base (G or A) preferable for the transcription start point of polIII on the 5′-terminal side, and a terminator sequence of polIII on the 3′-terminal side. Moreover, the DNA sequence comprises the restriction enzyme BglII recognition sequence (AGATCT) and the restriction enzyme HindII recognition sequence (AAGCTT), which are used in vector cloning, at the 5′-terminus and at the 3′-terminus, respectively.

An outline of the DNA sequences encoding individual guide RNAs is shown in Table 1.

TABLE 1

| | | | | | | | | | SEQ ID |
|---|---|---|---|---|---|---|---|---|---|
| | Names of guide RNAs | 5′- | Linker | ASR | ARR | Linker | Linker | 3′- | NO: |
| Comp. Ex. | ASR(24)-ARR(16)add | — | — | 24 nt | 16 nt | add | — | — | 28 |
| Ex. 1 | ASR(24)-ARR(16)add-3Gq | — | — | Rluc | | 20 nt | — | 3Gq | 19 |
| Ex. 1 | ASR(24)-ARR(16)add-BoxB | — | — | | | | UCU | BoxB | 20 |
| Ex. 1 | BoxB-ASR(24)-ARR(16)add-3Gq | BoxB | UCU | | | | — | 3Gq | 21 |
| Ex. 2 | U6 upstream-ASR(24)-ARR(16)add-U6 | U6 up | — | | | | — | U6 | 25 |
| | downstream | | | | | | | down | |
| Comp. Ex. | ASR(24)-ARR(16) | — | — | | 16 nt | — | — | — | 29 |
| Ex. 1 | ASR(24)-ARR(16)-3Gq | — | — | | | | — | 3Gq | 22 |
| Ex. 1 | ASR(24)-ARR(16)-BoxB | — | — | | | | UCU | BoxB | 23 |
| Ex. 1 | BoxB-ASR(24)-ARR(16)-3Gq | BoxB | UCU | | | | — | 3Gq | 24 |
| Ex. 2 | U6 upstream-ASR(24)-ARR(16)-U6 downstream | U6 up | — | | | | — | U6 | 26 |
| | | | | | | | | down | |
| Comp. Ex. | ASR(24)-ARR(40)add | — | — | | 40 nt | add | — | — | 30 |
| Comp. Ex. | ASR(24)-ARR(40)add-3Gq | — | — | | | 20 nt | — | 3Gq | 31 |
| Comp. Ex. | ASR(24)-ARR(40)add-BoxB | — | — | | | | UCU | BoxB | 32 |
| Comp. Ex. | BoxB-ASR(24)-ARR(40)add-3Gq | BoxB | UCU | | | | — | 3Gq | 33 |
| Comp. Ex. | U6 upstream-ASR(24)-ARR(40)add-U6 | U6 up | — | | | | — | U6 | 34 |
| | downstream | | | | | | | down | |
| Control | ASR(24)-ARR(40) | — | — | | 40 nt | — | — | — | 27 |
| Comp. Ex. | ASR(24)-ARR(40)-3Gq | — | — | | | | — | 3Gq | 35 |
| Comp. Ex. | ASR(24)-ARR(40)-BoxB | — | — | | | | UCU | BoxB | 36 |
| Comp. Ex. | BoxB-ASR(24)-ARR(40)-3Gq | BoxB | UCU | | | | — | 3Gq | 37 |
| Comp. Ex. | U6 upstream-ASR(24)-ARR(40)-U6 downstream | U6 up | — | | | | — | U6 | 38 |
| | | | | | | | | down | |

### Example 3

### Production of Reporter mRNA Expression Plasmid for Detecting Intracellular Target RNA-Editing Activity

Renilla luciferase (Rluc) mRNA was used as a reporter mRNA for detecting an intracellular target RNA-editing activity. The reporter Rluc mRNA (Rluc-W104X) for detecting an intracellular target RNA-editing activity was designed based on a Rluc mRNA sequence encoded by a Rluc gene incorporated in a psiCHECK-2 vector (Promega, Catalog No. C8021). Specifically, Rluc-W104X was designed such that the 311th G of UGG encoding Trp that is the 104th amino acid of Rluc was substituted with A, and that the translation termination codon (UAG) was encoded, instead of Trp. A Rluc-W104X expression plasmid was produced by substituting the DNA sequence (SEQ ID NO: 40) comprising the mutation point G311A with a psiCHECK-2 vector (Promega, Catalog No. C8021), using the restriction enzymes DraIII (on the 5′-terminal side) and AatII (on the 3′-terminal side). The thus obtained plasmid is referred to as “psiCHECK-2 Rluc-W104X.” The constructed psiCHECK-2 Rluc-W104X vector was amplified using the Escherichia coli strain DH5α or the Escherichia coli strain Stbl3 by the same method as that of Example 1.

### Example 4

### Production of ADAR2 Expression Plasmid

An ADAR2 expression plasmid was produced by inserting the DNA sequence (SEQ ID NO: 11) encoding ADAR2 (SEQ ID NO: 12) into the site of the restriction enzymes EcoRI (on the 5′-terminal side) and BamHI (on the 3′-terminal side) located downstream of the CMV-derived promoter of a pAAV-CMV vector contained in AAVpro(registered trademark) Helper Free System (Takara Bio, Inc., Catalog No. 6230), using the restriction enzymes EcoRI (on the 5′-terminal side) and BglII (on the 3′-terminal side). The obtained plasmid is referred to as “pAAV-CMV-ADAR2.” In the inserted DNA sequence, a Kozak sequence was disposed upstream the translation initiation codon, and a termination codon was disposed downstream of the ADAR2 gene. The constructed pAAV-CMV-ADAR2 plasmid was amplified using the Escherichia coli strain DH5α or the Escherichia coli strain Stbl3.

### Example 5

### Transfection

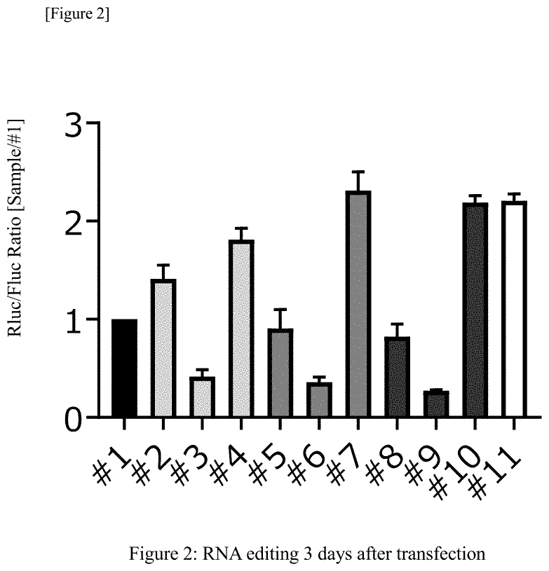

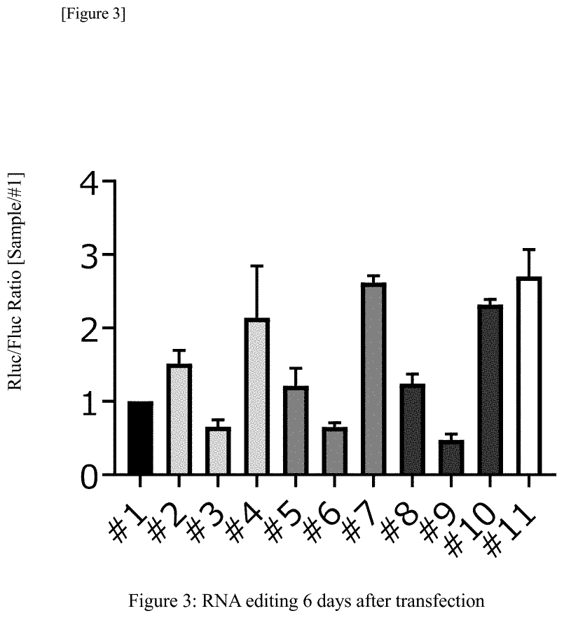

Transfection 1 (Transfection performed for luciferase assay whose results are shown in FIGS. 2 and 3, and for studies regarding RNA-editing efficiency according to Sanger sequencing whose results are shown in Table 2)

The human embryonic kidney-derived cell line HEK293 (ATCC Catalog No. CRL-1573) was cultured in a Dulbecco's Modified Eagle Medium (DMEM; Thermo Fisher Scientific, Catalog No. 10569-010) supplemented with 5% fetal bovine serum (FBS). The cultured cells were seeded on a 96-well plate at a cell density of 2.5×10⁴ cells/100 μL, and were then cultured in the presence of 5% CO₂ at 37° C. overnight. On the following day, the reporter expression plasmid (psiCHECK-2_Rluc-W104X) for detecting an intracellular target RNA-editing activity produced in Example 3, the ADAR2 expression plasmid (pAAV-CMV-ADAR2) produced in Example 4, and the guide RNA expression plasmids produced in Examples 1 and 2 (the after-mentioned three types of pSUPERneo_H1ADg and 1 type of pSUPERneo_U6ADg), and a carrier plasmid were mixed with one another at a weight ratio of 1:35:35:9, respectively, to result in a total amount of 100 ng/well. As such a carrier plasmid, pHelper Vector (Takara Bio, Inc., Catalog No. 6230) was used. Using Lipofectamine 3000 Transfection Reagent (Thermo Fisher Scientific, Catalog No. L3000015), the HEK293 cells were transfected with the above-mixed plasmid.

<Names of Guide RNAs Expressed by pSUPERneo_H1ADg or pSUPERneo_U6ADg>

ASR(24)-ARR(16)add-3Gq

ASR(24)-ARR(16)add-BoxB

U6 upstream-ASR(24)-ARR(16)add-U6 downstream

BoxB-ASR(24)-ARR(16)add-3Gq

For each plasmid, an experiment was carried out with every 3 wells. After completion of the transfection, the resulting cells were cultured in the presence of 5% CO₂ at 37° C., and on Days 3 and 6 of the culture, the cells were harvested, and were then subjected to the detection of an RNA-editing activity according to luciferase assay described in Example 6 and the studies of RNA-editing efficiency according to Sanger sequencing described in Example 7.

Guide RNA expression plasmids used as controls or comparative examples were also prepared in the same manner as that of Transfection 1, and were then transfected into HEK293 cells.

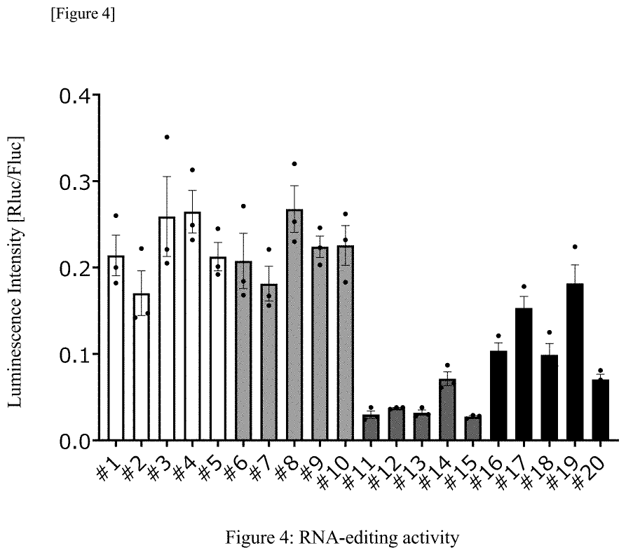

Transfection 2 (Transfection performed for luciferase assay whose results are shown in FIG. 4, and for studies regarding RNA-editing efficiency according to Sanger sequencing whose results are shown in Table 2)

The reporter expression plasmid (psiCHECK-_Rluc-W104X) for detecting an intracellular target RNA-editing activity, the ADAR2 expression plasmid (pAAV-CMV-ADAR2), and the 8 types of guide RNA expression plasmids produced in Examples 1 and (the after-mentioned 6 types of pSUPERneo_H1ADg and 2 types of pSUPERneo_U6ADg), and a carrier plasmid (pHelper Vector) were mixed with one another at a weight ratio of 1:30:40:9, respectively, to result in a total amount of 100 ng/well. Transfection of the mixed plasmid into the HEK293 cells were was carried out by the same method as that of Transfection 1. Besides, the cells were seeded on the plate at a cell density of 2.0×10⁴ cells/100 μL. After completion of the transfection, the cells were harvested on Day 3 of the culture.

<Names of Guide RNAs Expressed by pSUPERneo_H1ADg or pSUPERneo_U6ADg>

ASR(24)-ARR(16)add-3Gq

ASR(24)-ARR(16)add-BoxB

U6 upstream-ASR(24)-ARR(16)add-U6 downstream

BoxB-ASR(24)-ARR(16)add-3Gq

ASR(24)-ARR(16)-3Gq

ASR(24)-ARR(16)-BoxB

U6 upstream-ASR(24)-ARR(16)-U6 downstream

BoxB-ASR(24)-ARR(16)-3Gq

Guide RNA expression plasmids used as controls or comparative examples were also used in transfection in the same manner as that of Transfection 2.

### Example 6

### Detection of RNA-Editing Activity According to Luciferase Assay

The firefly luciferase luminescence intensity (Fluc) and the Renilla luciferase luminescence intensity (Rluc) of the cells transfected in Example 5 in each well were measured using Dual-Glo(registered trademark) Luciferase Assay System (Promega, Catalog No. E2940) and EnVision (PerkinElmer) according to protocols included therewith.

In FIG. 2 and FIG. 3, the Rluc/Fluc value was calculated to correct transfection efficiency among the samples, and the Rluc/Fluc value of each guide RNA sample was shown as a relative value to the Rluc/Fluc value of a guide RNA used as a control that was set to be 1. In the bar graph, the arithmetic mean of the 3 wells was indicated with an error bar and a standard deviation. The same tendency was shown from two times of trials, and the results of representative examples are shown in FIG. 2 (Day 3 of the culture) and FIG. 3 (Day 6 of the culture).

The correspondence relationships of individual numbers #1 to #11 and the names of guide RNAs, which are shown in FIG. 2 and FIG. 3, are shown below. The following names of guide RNAs correspond to the “Names of guide RNAs” shown in Table 1.
#1 ASR(24)-ARR(40)
#2 ASR(24)-ARR(40)-3Gq
#3 ASR(24)-ARR(40)add-3Gq
#4 ASR(24)-ARR(16)add-3Gq
#5 ASR(24)-ARR(40)-BoxB
#6 ASR(24)-ARR(40)add-BoxB
#7 ASR(24)-ARR(16)add-BoxB
#8 U6 upstream-ASR(24)-ARR(40)-U6 downstream
#9 U6 upstream-ASR(24)-ARR(40)add-U6 downstream
#10 U6 upstream-ASR(24)-ARR(16)add-U6 downstream
#11 BoxB-ASR(24)-ARR(16)add-3Gq

In FIG. 4, the arithmetic means of the Rluc/Fluc values obtained in independent 3 trials are shown using a bar graph. In addition, the plot indicates the arithmetic means of the Rluc/Fluc values of the samples in each trial, and the error bar indicates a standard deviation.

The correspondence relationships of individual numbers #1 to #20 and the names of guide RNAs, which are shown in FIG. 4, are shown below. The following names of guide RNAs correspond to the “Names of guide RNAs” shown in Table 1.
#1 ASR(24)-ARR(16)add
#2 ASR(24)-ARR(16)add-3Gq
#3 ASR(24)-ARR(16)add-BoxB
#4 BoxB-ASR(24)-ARR(16)add-3Gq
#5 U6 upstream-ASR(24)-ARR(16)add-U6 downstream
#6 ASR(24)-ARR(16)
#7 ASR(24)-ARR(16)-3Gq
#8 ASR(24)-ARR(16)-BoxB
#9 BoxB-ASR(24)-ARR(16)-3Gq
#10 U6 upstream-ASR(24)-ARR(16)-U6 downstream
#11 ASR(24)-ARR(40)add
#12 ASR(24)-ARR(40)add-3Gq
#13 ASR(24)-ARR(40)add-BoxB
#14 BoxB-ASR(24)-ARR(40)add-3Gq
#15 U6 upstream-ASR(24)-ARR(40)add-U6 downstream
#16 ASR(24)-ARR(40)
#17 ASR(24)-ARR(40)-3Gq
#18 ASR(24)-ARR(40)-BoxB
#19 BoxB-ASR(24)-ARR(40)-3Gq
#20 U6 upstream-ASR(24)-ARR(40)-U6 downstream

In the reporter (Rluc-W104X) for detecting an intracellular target RNA-editing activity, UGG encoding the 104th Trp of Rluc is substituted with the termination codon (UAG). Accordingly, if the editing of the A of the target RNA to I is induced in a cell by the ADAR protein and the guide RNA, the mutated translation termination codon is converted to Trp when it is translated from the mRNA, and the luminescence of Renilla luciferase is recovered. Specifically, it is shown that the higher the luminescence intensity, the higher the RNA-editing activity that can be obtained.

As shown from FIG. 2 to FIG. 4, a guide RNA comprising a functional nucleotide sequence and a short-chain ADAR-recruiting nucleotide sequence ARR(16) exhibited a higher RNA-editing activity than those of controls (i.e. #1 in FIGS. 2 and 3, and #16 in FIG. 4) (a guide RNA comprising the ADAR-recruiting nucleotide sequence ARR(40)), regardless of the presence or absence of a linker nucleotide sequence. On the other hand, when a functional nucleotide sequence was added to such a control via a linker nucleotide sequence, the RNA-editing activity was decreased, compared with the case of not using a linker nucleotide sequence.

### Example 7

### Studies Regarding RNA-Editing Efficiency according to Sanger Sequencing

The transfected cells obtained in Transfections 1 and 2 of Example 5 were each harvested, and total RNA was then extracted and purified from the harvested cells using QIAshredder (QIAGEN, Catalog No. 79656), RNeasy Mini Kit (QIAGEN, Catalog No. 74106), and RNase-free DNase Set (QIAGEN, Catalog No. 79254), according to protocols included therewith. The purified RNA was subjected to a reverse transcription reaction, using SuperScript(registered trademark) VILO cDNA Synthesis kit (Thermo Fisher Scientific, Catalog No. 11754-250), according to protocols included therewith, so as to obtain a cDNA. Using the obtained cDNA as a template and PrimeSTAR(registered trademark) GXL DNA Polymerase (Takara Bio, Inc., Catalog No. R050A), a PCR reaction was carried out employing the following primers for amplifying a fragment containing an edition point, according to protocols included therewith.

<Primers used in the Experiment Shown in Table 2>

Forward primer:
(SEQ ID NO: 41)
GGTAACGCTGCCTCCAGC
Reverse primer:
(SEQ ID NO: 42)
TGTAGTTGCGGACAATCTGG

<Primers used in the experiment shown in Table 3>

Forward primer:
(SEQ ID NO: 43)
CTCGCTGCAAGCAAATGAAC
Reverse primer:
(SEQ ID NO: 44)
TCGTCCCAGGACTCGATCAC

The PCR amplified fragment was purified using ExoSAP-IT Express PCR Cleanup Reagents (Thermo Fisher Scientific, Catalog No. 75001.200.UL) according to protocols included therewith, and the resulting product, together with the forward primer used in the PCR amplification, was subjected to a Sanger sequencing reaction using 3730xl (XL) DNA Analyzer (Applied Biosystems).

In Table 2, the DNA chromatogram (filename extension: .ab1) obtained in the Sanger sequencing reaction was analyzed using QSVanalyzer software (Bioinformatics, 2009, Vol. 25, pp. 3244-3250), and the abundance ratio of G (G/(G+A)×100), which was obtained when the signal strength of the guanine (G) of unedited Rluc-W104X was set at 0, the signal strength of the adenine (A) thereof was set at 1, the signal strength of G of Rluc was set at 1, and the signal strength of A thereof is set at 0, was defined as RNA-editing efficiency (%). The same tendency was shown from two times of trials, and the results of representative examples are shown in FIG. 2.

TABLE 2

| Functional | | RNA-editing | |
|---|---|---|---|
| nucleotide | | efficiency (%) | |
| sequences | Names of guide RNAs | Day 3 | Day 6 |
| Control | ASR(24)-ARR(40) | 40.7 | 53.4 |
| Gq | ASR(24)-ARR(40)-3Gq | 43.2 | 50.9 |
| | ASR(24)-ARR(40)add-3Gq | 17.1 | 28.7 |
| | ASR(24)-ARR(16)add-3Gq | 65.3 | 70.6 |
| BoxB | ASR(24)-ARR(40)-BoxB | 40.9 | 50.5 |
| | ASR(24)-ARR(40)add-BoxB | 22.0 | 38.8 |
| | ASR(24)-ARR(16)add-BoxB | 81.6 | 82.0 |
| U6 insert | U6 upstream-ASR(24)-ARR(40)- | 44.5 | 61.5 |
| | U6 downstream | | |
| | U6 upstream-ASR(24)-ARR(40) | 21.8 | 44.4 |
| | add-U6 downstream | | |
| | U6 upstream-ASR(24)-ARR(16)- | 55.7 | 66.1 |
| | U6 downstream | | |
| BoxB/Gq | BoxB-ASR(24)-ARR(16)add-3Gq | 71.0 | 70.1 |

The guide RNA used as a control (a guide RNA comprising an ADAR-recruiting nucleotide sequence ARR(40)) exhibited an RNA-editing efficiency of 40.7% on day 3 after the transfection. On the other hand, the guide RNA of the present invention (a guide RNA comprising a functional nucleotide sequence and a short-chain ADAR-recruiting nucleotide sequence ARR(16)) exhibited a high RNA-editing efficiency of approximately 60% to 80%.

In Table 3, the DNA chromatogram (filename extension: .ab1) obtained in the Sanger sequencing reaction was analyzed using the QSVanalyzer software, and the signal strength ratio of guanine (G) (G/(G+A)×100) was defined as an RNA-editing efficiency (%). The same tendency was shown from two times of trials, and the results of representative examples are shown below.

TABLE 3

| | Com- | | RNA- |
|---|---|---|---|
| | binations | Functional | editing |
| | of ARRs | nucleotide | efficiency |
| Names of guide RNAs | and linkers | sequences | (%) |
| ASR(24)-ARR(16)add | ARR(16)add | — | 65.6 |
| ASR(24)-ARR(16)add-3Gq | | 3Gq | 61.1 |
| ASR(24)-ARR(16)add-BoxB | | BoxB | 73.5 |
| BoxB-ASR(24)-ARR(16)add-3Gq | | BoxB/3Gq | 74.4 |
| U6 upstream-ASR(24)-ARR(16) | | U6 insert | 75.8 |
| add-U6 downstream | | | |
| ASR(24)-ARR(16) | ARR(16) | — | 68.4 |
| ASR(24)-ARR(16)-3Gq | | 3Gq | 62.8 |
| ASR(24)-ARR(16)-BoxB | | BoxB | 76.7 |
| BoxB-ASR(24)-ARR(16)-3Gq | | BoxB/3Gq | 70.6 |
| U6 upstream-ASR(24)-ARR(16)- | | U6 insert | 76.8 |
| U6 downstream | | | |
| ASR(24)-ARR(40)add | ARR(40)add | — | 26.6 |
| ASR(24)-ARR(40)add-3Gq | | 3Gq | 31.0 |
| ASR(24)-ARR(40)add-BoxB | | BoxB | 32.8 |
| BoxB-ASR(24)-ARR(40)add-3Gq | | BoxB/3Gq | 43.2 |
| U6 upstream-ASR(24)-ARR(40) | | U6 insert | 32.9 |
| add-U6 downstream | | | |
| ASR(24)-ARR(40) | ARR(40) | — | 44.6 |
| ASR(24)-ARR(40)-3Gq | | 3Gq | 43.2 |
| ASR(24)-ARR(40)-BoxB | | BoxB | 48.1 |
| BoxB-ASR(24)-ARR(40)-3Gq | | BoxB/3Gq | 63.7 |
| U6 upstream-ASR(24)-ARR(40)- | | U6 insert | 46.3 |
| U6 downstream | | | |

The guide RNA used as a control (ASR(24)-ARR(40)) (a guide RNA comprising an ADAR-recruiting nucleotide sequence ARR(40)) exhibited an RNA-editing efficiency of 44.6%. On the other hand, a guide RNA comprising a functional nucleotide sequence and a short-chain ADAR-recruiting nucleotide sequence ARR(16)) exhibited a high RNA-editing efficiency of approximately 60% to 75%, compared with the guide RNA used as a control.

### Example 8

### Studies Regarding RNA-Editing Efficiency Targeting Firefly Luciferase (Fluc) mRNA

Whether the guide RNA of the present invention exhibits a high RNA-editing efficiency even to a different target RNA was examined.

A guide RNA expression plasmid having, as an editing target, the 74th nucleotide adenosine in the coding region of firefly luciferase (Fluc) incorporated in a psiCHECK-2 vector (Promega, Catalog No. C8021) was produced in the same manner as those of Examples 1 and 2. ASRf(24) indicates an antisense nucleotide sequence consisting of 24 nucleotides complementary to a Fluc reporter mRNA comprising the adenosine as an editing target, and consists of an RNA nucleotide sequence encoded by a DNA nucleotide sequence from the nucleotide numbers 9 to 32 as set forth in SEQ ID NO: 45. The DNA sequences encoding the guide RNAs used in the production are as follows.

### <Names of Guide RNAs and Sequence Numbers of DNAs Encoding Guide RNAs>

ASRf(24)-ARR(16)add-3Gq: SEQ ID NO: 45

ASRf(24)-ARR(16)add-BoxB: SEQ ID NO: 46

U6 upstream-ASRf(24)-ARR(16)add-U6 downstream: SEQ ID NO: 47

The DNA sequence comprises a purine base (G or A) preferable for the transcription start point of polIII on the 5′-terminal side, and a terminator sequence of polIII on the 3′-terminal side. In addition, the DNA sequence comprises the sequence (AGATC) comprising the restriction enzyme BglII protruding end and the restriction enzyme HindIII recognition sequence (AAGCTT), which are used in vector cloning, at the 5′-terminus and at the 3′-terminus, respectively.

The obtained plasmids are collectively referred to as “pSUPERneo_H1ADg_ASRf24.”

Besides, guide RNA expression plasmids used as a control and as comparative examples were also produced in the same manner as that described above, using DNA sequences encoding guide RNAs (as shown below).

<Name of Guide RNA and Sequence Number of DNA Encoding Guide RNA (control)>

ASRf(24)-ARR(40): SEQ ID NO: 48

### <Names of Guide RNAs and Sequence Numbers of DNAs Encoding Guide RNAs (Comparative Examples)>

ASRf(24)-ARR(16)add: SEQ ID NO: 49

ASRf(24)-ARR(16): SEQ ID NO: 50

The DNA sequence comprises a purine base (G or A) preferable for the transcription start point of polIII on the 5′-terminal side, and a terminator sequence of polIII on the 3′-terminal side. In addition, the DNA sequence comprises the sequence (AGATC) comprising the restriction enzyme BglII protruding end and the restriction enzyme HindIII recognition sequence (AAGCTT), which are used in vector cloning, at the 5′-terminus and at the 3′-terminus, respectively.

In the same manner as that of Example 5 (Transfection 2), HEK293 cells were transfected with the Fluc reporter expression plasmid (psiCHECK-2 (Promega, C8021)), the ADAR2 expression plasmid (pAAV-CMV-ADAR2) produced in Example 4, and the above-described guide RNA expression plasmid (pSUPERneo_H1ADg_ASRf24). The guide RNA expression plasmids used as controls and comparative examples were also used in the transfection.

A PCR reaction was carried out in the same manner as that of Example 7 (in the case shown in Table 3), and a Sanger sequencing reaction was then carried out using sequencing primers. The RNA-editing efficiency targeting the Fluc reporter mRNA was calculated from the chromatogram of the Sanger sequencing reaction. The primers used in the PCR reaction and the Sanger sequencing reaction of the present test are shown below.

Forward primer Fluc:
(SEQ ID NO: 51)
GGCCGATGCTAAGAACATTAAGAAG
Reverse primer Fluc:
(SEQ ID NO: 52)
CCACGGTAGGCTGAGAAATG
Sequencing primer:
(SEQ ID NO: 53)
CTCCGATGAACAGGGCGC

The obtained results are shown in Table 4.

TABLE 4

| | Combinations | | RNA- |
|---|---|---|---|
| | of | Functional | editing |
| | ARRs and | nucleotide | efficiency |
| Names of guide RNAs | linkers | sequences | (%) |
| ASRf(24)-ARR(16)add | ARR(16)add | — | 8.1 |
| ASRf(24)-ARR(16)add-3Gq | | 3Gq | 15.2 |
| ASRf(24)-ARR(16)add-BoxB | | BoxB | 4.3 |
| U6 upstream-ASRf(24)- | | U6 insert | 3.1 |
| ARR(16)add-U6 downstream | | | |
| ASRf(24)-ARR(16) | ARR(16) | — | 9.0 |
| ASRf(24)-ARR(40) | ARR(40) | — | 1.8 |

The guide RNA used as a control (ASRf(24)-ARR(40)) (a guide RNA comprising an ADAR-recruiting nucleotide sequence ARR(40)) exhibited an RNA-editing efficiency of 1.8% on 3 days after the transfection. On the other hand, the guide RNA comprising a functional nucleotide sequence and a short-chain ADAR-recruiting nucleotide sequence ARR(16) exhibited a high RNA-editing efficiency of approximately 3% to 15%, compared with the guide RNA used as a control.

### Reference Example 1

### Studies Regarding Stability of Guide RNA In Vitro

The stability of a guide RNA in vitro can be evaluated by making a comparison in terms of the remaining amount of the synthesized guide RNA. As the remaining amount of a guide RNA of interest increases, it means that the stability of the guide RNA is high.

Specifically, a guide RNA is synthesized by in vitro transcription from a double-stranded DNA, the obtained guide RNA is then treated with exoribonuclease, and the remaining guide RNA is then quantified.

The double-stranded DNA is not particularly limited, as long as the guide RNA of the present invention can be synthesized by in vitro transcription from the double-stranded DNA. The double-stranded DNA may be, for example, a double-stranded DNA comprising a region encoding a guide RNA downstream of a promoter region. It is also possible to add a sequence enhancing transcription efficiency to the double-stranded DNA. For example, it is possible to add GG or GGG to the 5′-terminus of an RNA sequence to be transcribed. The promoter region is not particularly limited, either, and for example, a region comprising a T7 RNA polymerase, T3 RNA polymerase, or SP6 RNA polymerase-binding region can be used.

The method of producing a double-stranded DNA to be used in the in vitro transcription is not particularly limited. For example, a double-stranded DNA obtained by synthesizing two oligo DNAs having a complementary relationship to each other and then subjecting the two oligo DNAs to an ordinary annealing reaction, and a double-stranded DNA produced from a plasmid DNA, etc. used as a template according to a PCR method, can be used. Depending on the sequence of a double-stranded DNA, such a double-stranded DNA can be prepared according to a restriction enzyme treatment, a Fill-in reaction involving Klenow Fragment, and the like. In order to obtain a double-stranded DNA having such a purity that can be used in the in vitro transcription reaction, the produced double-stranded DNA is separated and/or purified according to, for example, agarose gel electrophoresis, polyacrylamide gel electrophoresis, phenol/chloroform extraction, or ethanol precipitation, and the resultant can be then used in the in vitro transcription.

The in vitro transcription from the double-stranded DNA is not particularly limited, and it can be carried out using, for example, T7-Scribe Standard RNA IVT KIT (CELLSCRIPT, Catalog No. C-AS2607), MAXIscript T7 Transcription Kit (Thermo Fisher, Catalog No. AM1312), and T7 RNA polymerase (Takara Bio, Inc., Catalog No. 2540A), according to protocols included therewith. Purification of an RNA from the reaction solution obtained after completion of the in vitro transcription can be carried out according to an ordinary method. For instance, the reaction solution is treated with DNase, an RNA is then purified by phenol/chloroform extraction and ethanol precipitation, and the purified RNA is further separated according to gel electrophoresis, so that an RNA can be purified from a band at a desired position. As such gel electrophoresis, denaturing polyacrylamide gel electrophoresis can be, for example, used. For purification of the RNA from the gel, filter filtration and gel filtration can be used.

As exoribonuclease used in the exoribonuclease treatment of a guide RNA, for example, RNaseR (Lucigen, Catalog No. RNR07250) or RNaseR (BioVision, Catalog No. M1228-500) can be used. The buffers and conditions used in the exoribonuclease treatment are not particularly limited. For instance, 0.1 to 10 U RNaseR can be used with respect to 250 ng of a guide RNA, and the reaction can be carried out in 1× RNase R Reaction Buffer (20 mM Tris-HCl (pH8.0), 100 mM KCl, and 0.1 mM MgCl₂) under conditions of 37° C. and 10 minutes to 2 hours. As described later, in the case of using a radioisotope (RI) labeled RNA or a fluorescently labeled RNA, the RNA can also be treated using a cell extraction solution, instead of exoribonuclease.

Quantification of the RNA remaining after the exoribonuclease treatment can be carried out according to an ordinary method. For example, the solution obtained after completion of the exoribonuclease treatment reaction is separated by gel electrophoresis, and the gel is then stained, so that a remaining RNA can be quantified. Herein, for the gel electrophoresis, denaturing polyacrylamide gel electrophoresis can be used. For the staining of the gel, ethidium bromide or SYBR Gold Nucleic Acid Gel Stain (Thermo Fisher, Catalog No. S11494) can be used. As another method of quantifying a remaining RNA, a bioanalyzer (manufactured by Agilent; 2100 BioAnalyzer) can also be used.

As another method of quantifying an RNA remaining after the exoribonuclease treatment, a method of quantifying an RNA labeled with RI, fluorescence, etc. can also be applied. The RI used in the labeling is not particularly limited, as long as it is able to label the RNA. For example, γ-³²P-ATP can be used. As a labeling method with RI, for example, the 5′-terminus of an RNA is dephosphorylated using a dephosphorylation enzyme, and the RNA is then purified by phenol/chloroform extraction, and thereafter, a phosphorylation reaction can be carried out using polynucleotide kinase in the presence of [γ-³²P]-ATP. As such a dephosphorylation enzyme, for example, Antarctic Phosphatase (New England Biolabs, Catalog No. M0289S) can be used. As ATP, for example, [γ-³²P]-ATP (PerkinElmer, Catalog No. BLU002Z250UC) can be used. As polynucleotide kinase, for example, T4 polynucleotide kinase (New England Biolabs, Catalog No. M0201S) can be used. The fluorescent dye used in the labeling is not particularly limited, as long as it is able to label the RNA. The fluorescently labeling method may be, for example, a method of labeling the 5′-terminus, and 5′ EndTag™ Nucleic Acid Labeling System (Vector Laboratories, Catalog No. MB-9001) can be used. The thus RI-labeled or fluorescently labeled RNA is subjected to an exoribonuclease treatment according to the same method as that described above, and after completion of the reaction, the solution is separated by gel electrophoresis. After completion of the separation, radioactivity or fluorescence intensity on the gel is detected using an image analyzer, so that the remaining RNA can be quantified. As such an image analyzer, for example, Fluoro Image Analyzer (Fujifilm, Catalog No. FLA-7000) can be used.

An example of a detailed method of studying the stability of a guide RNA in vitro by making a comparison in terms of the remaining amount of the guide RNA will be described below, but the example is not limited thereto.

Using, as a template, a plasmid DNA (wherein, for example, the guide RNA expression plasmid (pSUPERneo_H1 gRNA or pSUPERneo_U6_RNA) described in Example 1 or 2 can be used), a PCR reaction is carried out using a T7 RNA polymerase-binding sequence, a sequence enhancing transcription activity, a forward primer comprising a sequence encoding a part of a guide RNA, a reverse primer comprising a sequence encoding a part of the guide RNA, and Prime Star GXL (Takara Bio, Inc., Catalog No. R050B). Thereafter, the amplified PCR product is purified by phenol/chloroform extraction and ethanol precipitation to obtain a template DNA. Using the obtained DNA as a template, in vitro transcription is carried out (wherein the reaction conditions are, for example, 37° C. and 3 hours) employing T7-Scribe Standard RNA IVT KIT (manufactured by CELLSCRIPT) according to protocols included therewith, so as to synthesize an RNA. Thereafter, DNase is added to perform a reaction of decomposing the template DNA, and an RNA is then purified by phenol/chloroform extraction and ethanol precipitation. After that, the obtained RNA is purified by 8 M Urea PAGE (8%), and is then extracted by crushing and/or immersion. The extract is purified using a 0.22-μm filter and gel filtration, thereby preparing various types of guide RNAs.

The guide RNA is subjected to a dephosphorylation reaction (e.g. 37° C., 1 hour), using Antarctic Phosphatase (New England Biolabs, Catalog No. M0289S). After completion of the reaction, the RNA is purified by phenol-chloroform extraction and ethanol precipitation.

[γ-³²P]-ATP (PerkinElmer#BLU002Z250UC) is added to the RNA after completion of the dephosphorylation, and a phosphorylation reaction (e.g. 37° C., 30 minutes) is then carried out using 1 U/μl (final concentration) T4 polynucleotide kinase (New England Biolabs, Catalog No. M0201S). After completion of the reaction, the RNA is purified by ethanol precipitation. The RNA is dissolved in 80% formamide, and is then cut out by 8 M Urea 8% polyacrylamide gel electrophoresis (PAGE), and the RNA is then purified by the same method as that described above. The radioactivity of the obtained RNA is measured using a liquid scintillation counter (PerkinElmer, Catalog No. Tri-Carb 2910 TR).

For example, 0.1 to 10 U RNase R is added to the radiolabeled guide RNA, and a decomposition reaction is then carried out (e.g. 37° C., 10 minutes to 2 hours). After completion of the reaction, the solution is separated by 8 M Urea 8% polyacrylamide gel electrophoresis (PAGE). The surface of the gel is covered with a wrap, and a Fuji imaging plate (Fujifilm, Catalog No.) is then placed thereon, followed by performing light exposure for 6 hours or more. After completion of the light exposure, the imaging plate is analyzed using an image analyzer (Fujifilm, Catalog No. FLA-7000). The remaining RNA is quantified from the obtained band.

### Reference Example 2

### Studies Regarding Stability of Guide RNA in Cells

The stability of a guide RNA in cells can be evaluated by making a comparison in terms of the amount of a guide RNA remaining after inhibition of the transcription. As the amount of a remaining guide RNA of interest increases, it means that the guide RNA has high stability.

Specifically, after a guide RNA expression plasmid has been transfected into the cultured cells and then the guide RNA has been sufficiently expressed therein, the transcription of the guide RNA is inhibited by a transcription inhibitor for a certain period of time. Thereafter, the RNA is extracted and purified from the cells, and the remaining guide RNA is then quantified. In another aspect, a guide RNA expression plasmid is transfected into the cultured cells, and a newly transcribed RNA is pulse-labeled. Thereafter, the labeled RNA is recovered, and the remaining guide RNA is then quantified.

The guide RNA expression plasmid is not particularly limited, as long as it is able to express the guide RNA of the present invention. For example, the guide RNA expression plasmid (pSUPERneo_H1ADg or pSUPERneo_U6ADg) described in Examples 1 and 2 can be used. The reporter expression plasmid for detecting an intracellular target RNA-editing activity (psiCHECK-2_Rluc-W104X) described in Example 3 and the ADAR2 expression plasmid (pAAV-CMV-ADAR2) described in Example 4 may be used in the transfection simultaneously with the guide RNA expression plasmid.

The cultured cells are not particularly limited, as long as they are able to express the guide RNA. For example, HEK293 cells can be used.

The guide RNA expression plasmid can be transfected into the cultured cells according to an ordinary method. The transfection method is not particularly limited, and the transfection can be carried out, for example, using Lipofectamine 3000 Transfection Reagent, according to protocols included therewith. The concentration of the guide RNA expression plasmid used in the transfection can be set to be an appropriate concentration, depending on the cultured cells.

The transcription inhibitor is not particularly limited. For example, actinomycin D (Wako Pure Chemical Industries, Ltd., Catalog No. 010-21263), α-amanitin (Wako Pure Chemical Industries, Ltd., Catalog No. 010-22961), etc. can be used. It has been known that actinomycin D binds to a double-stranded DNA and inhibits transcription by an RNA polymerase. It is also possible to use a Tet-inducible promoter, etc., instead of such a transcription inhibitor, so as to transiently express a guide RNA from a guide RNA expression plasmid.

It requires, for example, 24 hours after the transfection, in order to reach a state in which a guide RNA has been sufficiently expressed after the transfection of a guide RNA expression plasmid into the cultured cells, but the required time is not limited thereto.

After addition of the transcription inhibitor, the transfected cells are harvested at any given timing, and the RNA is then purified. Such any given timing for harvesting the cells can be determined, depending on a period of time, in which a highly stable RNA used as a positive control remains. Such any given timing for recovering the cells is not particularly limited, and it may be 30 minutes to 16 hours after addition of the transcription inhibitor.

Extraction and purification of a total RNA comprising the guide RNA from the harvested cells can be carried out according to an ordinary method. The method of extracting the RNA is not particularly limited. For example, a microRNA extraction/purification kit, such as miRNeasy Mini Kit (QIAGEN, Catalog No. 217004), or mirVana miRNA Isolation Kit (Thermo Fisher Scientific, Catalog No. AM1560), or NucleoSpin miRNA (Takara Bio, Inc., Catalog No. U0971B), can be used. When DNA is removed from the extracted total RNA, a kit such as RNase-free DNase Set can be used.

Quantification of the RNA can be carried out according to an ordinary method. For example, using cDNA produced from the extracted RNA as a template, a PCR reaction is carried out according to an ordinary method, and a guide RNA of interest can be quantified. The method of producing cDNA is not particularly limited. For example, poly A is added to a small RNA using Mir-X miRNA First-Strand Synthesis Kit (Takara Bio, Inc., Catalog No. Z8315N) according to protocols included therewith, and thereafter, a reverse transcription reaction is carried out to obtain cDNA. The method of quantifying a guide RNA according to a real-time PCR reaction is not particularly limited. Using the obtained cDNA as a template, a real-time PCR reaction is carried out employing QuantStudio 12K Flex Real-Time PCR System (Thermo Fisher Scientific), and using Mir-miRNA qRT-PCR TB Green(registered trademark) Kit (Takara Bio, Inc., Catalog No. Z8316N) according to protocols included therewith, so that the guide RNA is quantified. The forward primer used in the real-time PCR reaction can be designed based on the sequence of the guide RNA to be quantified, according to a method known to those skilled in the art.

In another aspect, a newly transcribed RNA is pulse-labeled, and the labeled RNA is then recovered, and thereafter, the remaining guide RNA can be quantified. The pulse-labeling reagent is not particularly limited, and for example, 5-bromouridine (BrU) can be used. Specifically, an RNA that is newly generated in cells can be labeled with BrU, using BRIC Kit (MBL Life Sciences, Catalog No. RN1007) according to protocols included therewith. The labeled RNA is recovered by an immunoprecipitation method, and the amount of the guide RNA is quantified according to the above-described PCR method.

An example of a detailed method of studying the stability of a guide RNA in cells by making a comparison in terms of the remaining amount of the guide RNA after inhibition of the transcription will be described below, but the example is not limited thereto.

The reporter expression plasmid for detecting an intracellular target RNA-editing activity (psiCHECK-_Rluc-W104X), the ADAR2 expression plasmid (pAAV-CMV-ADAR2), and the guide RNA expression plasmid (pSUPERneo_H1ADg or pSUPERneo_U6ADg) (all of which are described in Examples 1 to 4) are transfected into HEK293 cells, using Lipofectamine 3000 Transfection Reagent according to protocols included therewith. Twenty four hours later, a treatment with actinomycin D is carried out. It has been known that actinomycin D binds to a double-stranded DNA and inhibits transcription by an RNA polymerase. The transfected cells are harvested until 30 minutes to 16 hours after inhibition of the transcription, and then, a total RNA comprising the guide RNA is extracted and purified from the harvested cells, using miRNeasy Mini Kit and RNase-free DNase Set, according to protocols included therewith. With regard to the obtained RNA, poly A is added to a small RNA, using Mir-X miRNA First-Strand Synthesis Kit (Takara Bio, Inc., Catalog No. Z8315N), according to protocols included therewith, and thereafter, a reverse transcription reaction is carried out to obtain cDNA. Using the obtained cDNA as a template, a real-time PCR reaction is carried out employing QuantStudio™ 12K Flex Real-Time PCR System (Thermo Fisher Scientific), and using Mir-X miRNA qRT-PCR TB Green (registered trademark) Kit according to protocols included therewith, so that the guide RNA is quantified. The forward primer used in the real-time PCR reaction is designed to have a sequence homologous to the antisense nucleotide sequence of the nucleic acid encoding the guide RNA.

### INDUSTRIAL APPLICABILITY

The guide RNA of the present invention is useful for editing a target RNA. A pharmaceutical composition comprising the guide RNA, a nucleic acid encoding the guide RNA, the expression vector of the present invention, or the polypeptide of the present invention, is useful in that it can be used for various diseases including hereditary diseases as typical examples, which can be prevented or treated by editing a target RNA.

### REFERENCE SIGNS LIST

1: Guide RNA, 10: Antisense nucleotide sequence, 20: Short-chain ADAR-recruiting nucleotide sequence, 30: Functional nucleotide sequence, 40: Target RNA sequence, 50: ADAR, 60: Linker

### Sequence Listing Free Text

SEQ ID NOs: 1 to 10: Synthetic RNAs
SEQ ID NOs: 13 to 53: Synthetic DNAs

1. A guide RNA for editing a target RNA sequence, comprising:
an antisense nucleotide sequence complementary to a portion of the target RNA sequence, a short-chain ADAR-recruiting nucleotide sequence, and at least one functional nucleotide sequence.

2. The guide RNA according to claim 1, wherein the antisense nucleotide sequence, the short-chain ADAR-recruiting nucleotide sequence, and the at least one functional nucleotide sequence are connected with one another, in this order, from the 5′-terminal side to the 3′-terminal side.

3. The guide RNA according to claim 2, wherein at least one functional nucleotide sequence is further connected with the 5′-terminal side of the antisense nucleotide sequence.

4. The guide RNA according to claim 1, further comprising a linker nucleotide sequence.

5. The guide RNA according to claim 4, wherein at least one functional nucleotide sequence is connected with the short-chain ADAR-recruiting nucleotide sequence via the linker nucleotide sequence.

6. The guide RNA according to claim 5, wherein the antisense nucleotide sequence, the short-chain ADAR-recruiting nucleotide sequence, the linker nucleotide sequence, and the at least one functional nucleotide sequence are connected with one another, in this order, from the 5′-terminal side to the 3′-terminal side.

7. The guide RNA according to claim 6, wherein at least one functional nucleotide sequence is further connected with the 5′-terminal side of the antisense nucleotide sequence.

8. The guide RNA according to claim 1, wherein the functional nucleotide sequence is a nucleotide sequence associated with the intracellular stabilization and/or intracellular localization of the guide RNA.

9. The guide RNA according claim 1, wherein the functional nucleotide sequence(s) are one or more nucleotide sequences selected from a nucleotide sequence that forms a G-quadruplex structure and a nucleotide sequence that forms a stem-loop structure.

10. The guide RNA according to claim 1, wherein the functional nucleotide sequence is a U6 snRNA sequence.

11. The guide RNA according to claim 9, wherein the functional nucleotide sequence is a nucleotide sequence that forms a 1 to 4 layered G-quadruplex structure.

12. The guide RNA according to claim 9, wherein the functional nucleotide sequence is a nucleotide sequence that forms a stem-loop structure.

13. The guide RNA according to claim 12, wherein the nucleotide sequence that forms a stem-loop structure is a nucleotide sequence derived from a BoxB sequence.

14. The guide RNA according to claim 1, wherein the functional nucleotide sequence consists of the nucleotide sequence as set forth in SEQ ID NO: 6, 7, 8, or 9, or a nucleotide sequence comprising a deletion, substitution, insertion and/or addition of 1 to 3 nucleotides with respect to the nucleotide sequence as set forth in SEQ ID NO: 6, 7, 8, or 9, and having functions.

15. The guide RNA according to claim 1, wherein the short-chain ADAR-recruiting nucleotide sequence is a nucleotide sequence consisting of 14 to 34 nucleotides.

16. The guide RNA according to claim 15, wherein the short-chain ADAR-recruiting nucleotide sequence is a sequence consisting of 16 nucleotides.

17. The guide RNA according to claim 15, wherein the loop portion in the stem-loop structure of the short-chain ADAR-recruiting nucleotide sequence is a nucleotide sequence consisting of 4 or 5 nucleotides.

18. The guide RNA according to claim 15, wherein the stem portion in the stem-loop structure of the short-chain ADAR-recruiting nucleotide sequence has nucleotides that form a mismatched base pair.

19. The guide RNA according to claim 1, wherein the short-chain ADAR-recruiting nucleotide sequence consists of the nucleotide sequence as set forth in SEQ ID NO: 1, 2, 3, or 4, or a nucleotide sequence comprising a deletion, substitution, insertion and/or addition of 1 to 3 nucleotides with respect to the nucleotide sequence as set forth in SEQ ID NO: 1, 2, 3, or 4, and forming a stem-loop structure.

20. The guide RNA according to claim 4, wherein the linker nucleotide sequence is a nucleotide sequence consisting of 15 to 25 nucleotides.

21. A target RNA sequence-editing system, comprising the guide RNA according to claim 1.

22. A nucleic acid encoding the guide RNA according to claim 1.

23. An expression vector, comprising a nucleic acid encoding the guide RNA according to claim 1.

24. The expression vector according to claim 23, further comprising a nucleic acid encoding ADAR.

25. A host cell, into which the expression vector according to claim 23 or 24 is introduced.

26. A method for producing an expression vector, comprising a step of culturing the host cell according to claim 25.

27. A method for preventing or treating any given disease, to which a modification of one or more adenosine or cytidine residues in the target RNA provides an advantageous change, wherein the method comprises a step of administering to a patient, a preventively effective amount or therapeutically effective amount of the expression vector according to claim 23 and a pharmaceutically acceptable excipient.

28. A method for preventing or treating any given disease, to which a modification of one or more adenosine or cytidine residues in the target RNA provides an advantageous change, wherein the method comprises a step of administering to a patient, a preventively effective amount or therapeutically effective amount of the expression vector according to claim 23, an expression vector containing a nucleic acid encoding ADAR, and a pharmaceutically acceptable excipient.

29. The method according to claim 27, wherein the ADAR is human ADAR1 or human ADAR2.

30. The method according to claim 28, wherein the nucleic acid encoding ADAR is a nucleic acid consisting of a nucleotide sequence encoding the amino acid sequence as set forth in SEQ ID NO: 12, or a nucleotide sequence encoding a polypeptide that consists of an amino acid sequence having an identity of 90% or more to the amino acid sequence as set forth in SEQ ID NO: 12 and has an adenosine deaminase activity.

31. A method for editing a target RNA, comprising: (i) a step of introducing the guide RNA according to claim 1 or the nucleic acid according to claim 22 into a cell; (ii) a step of recruiting ADAR by the guide RNA; (iii) a step of forming a complex of a target RNA sequence and the guide RNA; (iv) a step of converting the adenosine of the target RNA sequence to inosine by the ADAR recruited by the guide RNA.


SEQUENCE LISTING (53 sequences)

SEQ ID NO: 1 - RNA, 14 nt, Artificial
  Synthetic RNA
ggguguucgcaccu

SEQ ID NO: 2 - RNA, 16 nt, Artificial
  Synthetic RNA
gggugguucgccaccu

SEQ ID NO: 3 - RNA, 24 nt, Artificial
  Synthetic RNA
ggguggaauauucguaucccaccu

SEQ ID NO: 4 - RNA, 34 nt, Artificial
  Synthetic RNA
ggguggaauaguauauucguauaguaucccaccu

SEQ ID NO: 5 - RNA, 40 nt, Artificial
  Synthetic RNA
ggguggaauaguauaccauucgugguauaguaucccaccc

SEQ ID NO: 6 - RNA, 24 nt, Artificial
  Synthetic RNA
ggguuuggguuuggguuuggguuu

SEQ ID NO: 7 - RNA, 24 nt, Artificial
  Synthetic RNA
agagggcccugaagagggcccuuu

SEQ ID NO: 8 - RNA, 33 nt, Artificial
  Synthetic RNA
gugcucgcuucggcagcacauauacuagucgac

SEQ ID NO: 9 - RNA, 26 nt, Artificial
  Synthetic RNA
ucuagagcggacuucgguccgcuuuu

SEQ ID NO: 10 - RNA, 20 nt, Artificial
  Synthetic RNA
caguccuggaaacagggacc

SEQ ID NO: 11 - DNA, 2127 bp, Homo sapiens
  CDS (16)..(2118)
gaattcgccgccaccatggatatagaagatgaagaaaacatgagttccagcagcactgatgtgaaggaaaaccgcaatctggacaacgtgtcccccaaggatggcagcacacctgggcctggcgagggctctcagctctccaatgggggtggtggtggccccggcagaaagcggcccctggaggagggcagcaatggccactccaagtaccgcctgaagaaaaggaggaaaacaccagggcccgtcctccccaagaacgccctgatgcagctgaatgagatcaagcctggtttgcagtacacactcctgtcccagactgggcccgtgcacgcgcctttgtttgtcatgtctgtggaggtgaatggccaggtttttgagggctctggtcccacaaagaaaaaggcaaaactccatgctgctgagaaggccttgaggtctttcgttcagtttcctaatgcctctgaggcccacctggccatggggaggaccctgtctgtcaacacggacttcacatctgaccaggccgacttccctgacacgctcttcaatggttttgaaactcctgacaaggcggagcctcccttttacgtgggctccaatggggatgactccttcagttccagcggggacctcagcttgtctgcttccccggtgcctgccagcctagcccagcctcctctccctgtcttaccaccattcccacccccgagtgggaagaatcccgtgatgatcttgaacgaactgcgcccaggactcaagtatgactttctctccgagagcggggagagccatgccaagagcttcgtcatgtctgtggtcgtggatggtcagttctttgaaggctcggggagaaacaagaagcttgccaaggcccgggctgcgcagtctgccctggccgccatttttaacttgcacttggatcagacgccatctcgccagcctattcccagtgagggtcttcagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtgaggggacgattccagtgcgctccaatgcgagcatccaaacgtgggacggggtgctgcaaggggagcggctgctcaccatgtcctgcagtgacaagattgcacgctggaacgtggtgggcatccagggatccctgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgccctgaagatcc

SEQ ID NO: 12 - protein, 701 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 13 - DNA, 16 bp, Artificial
  Synthetic DNA
gggtggttcgccacct

SEQ ID NO: 14 - DNA, 20 bp, Artificial
  Synthetic DNA
cagtcctggaaacagggacc

SEQ ID NO: 15 - DNA, 24 bp, Artificial
  Synthetic DNA
gggtttgggtttgggtttgggttt

SEQ ID NO: 16 - DNA, 24 bp, Artificial
  Synthetic DNA
agagggccctgaagagggcccttt

SEQ ID NO: 17 - DNA, 33 bp, Artificial
  Synthetic DNA
gtgctcgcttcggcagcacatatactagtcgac

SEQ ID NO: 18 - DNA, 26 bp, Artificial
  Synthetic DNA
tctagagcggacttcggtccgctttt

SEQ ID NO: 19 - DNA, 105 bp, Artificial
  Synthetic DNA
agatctggggaaggttcagcagctcgaaccaaggggtggttcgccacctcagtcctggaaacagggaccgggtttgggtttgggtttgggtttttttttaagctt

SEQ ID NO: 20 - DNA, 108 bp, Artificial
  Synthetic DNA
agatctggggaaggttcagcagctcgaaccaaggggtggttcgccacctcagtcctggaaacagggacctctagagggccctgaagagggccctttttttttaagctt

SEQ ID NO: 21 - DNA, 132 bp, Artificial
  Synthetic DNA
agatctgggagagggccctgaagagggcccttttctgaaggttcagcagctcgaaccaaggggtggttcgccacctcagtcctggaaacagggaccgggtttgggtttgggtttgggtttttttttaagctt

SEQ ID NO: 22 - DNA, 85 bp, Artificial
  Synthetic DNA
agatctggggaaggttcagcagctcgaaccaaggggtggttcgccacctgggtttgggtttgggtttgggtttttttttaagctt

SEQ ID NO: 23 - DNA, 88 bp, Artificial
  Synthetic DNA
agatctggggaaggttcagcagctcgaaccaaggggtggttcgccaccttctagagggccctgaagagggccctttttttttaagctt

SEQ ID NO: 24 - DNA, 112 bp, Artificial
  Synthetic DNA
agatctgggagagggccctgaagagggcccttttctgaaggttcagcagctcgaaccaaggggtggttcgccacctgggtttgggtttgggtttgggtttttttttaagctt

SEQ ID NO: 25 - DNA, 139 bp, Artificial
  Synthetic DNA
agatctgggtgctcgcttcggcagcacatatactagtcgacgaaggttcagcagctcgaaccaaggggtggttcgccacctcagtcctggaaacagggacctctagagcggacttcggtccgcttttttttttaagctt

SEQ ID NO: 26 - DNA, 120 bp, Artificial
  Synthetic DNA
agatctggggtgctcgcttcggcagcacatatactagtcgacgaaggttcagcagctcgaaccaaggggtggttcgccaccttctagagcggacttcggtccgcttttttttttaagctt

SEQ ID NO: 27 - DNA, 85 bp, Artificial
  Synthetic DNA
agatctggggaaggttcagcagctcgaaccaaggggtggaatagtataccattcgtggtatagtatcccacccttttttaagctt

SEQ ID NO: 28 - DNA, 81 bp, Artificial
  Synthetic DNA
agatctggggaaggttcagcagctcgaaccaaggggtggttcgccacctcagtcctggaaacagggaccttttttaagctt

SEQ ID NO: 29 - DNA, 61 bp, Artificial
  Synthetic DNA
agatctggggaaggttcagcagctcgaaccaaggggtggttcgccacctttttttaagctt

SEQ ID NO: 30 - DNA, 105 bp, Artificial
  Synthetic DNA
agatctggggaaggttcagcagctcgaaccaaggggtggaatagtataccattcgtggtatagtatcccaccccagtcctggaaacagggaccttttttaagctt

SEQ ID NO: 31 - DNA, 129 bp, Artificial
  Synthetic DNA
agatctggggaaggttcagcagctcgaaccaaggggtggaatagtataccattcgtggtatagtatcccaccccagtcctggaaacagggaccgggtttgggtttgggtttgggtttttttttaagctt

SEQ ID NO: 32 - DNA, 135 bp, Artificial
  Synthetic DNA
agatctggggaaggttcagcagctcgaaccaaggggtggaatagtataccattcgtggtatagtatcccaccccagtcctggaaacagggacctctagagggccctgaagagggcccttttttttttttaagctt

SEQ ID NO: 33 - DNA, 156 bp, Artificial
  Synthetic DNA
agatctgggagagggccctgaagagggcccttttctgaaggttcagcagctcgaaccaaggggtggaatagtataccattcgtggtatagtatcccaccccagtcctggaaacagggaccgggtttgggtttgggtttgggtttttttttaagctt

SEQ ID NO: 34 - DNA, 163 bp, Artificial
  Synthetic DNA
agatctgggtgctcgcttcggcagcacatatactagtcgacgaaggttcagcagctcgaaccaaggggtggaatagtataccattcgtggtatagtatcccaccccagtcctggaaacagggacctctagagcggacttcggtccgcttttttttttaagctt

SEQ ID NO: 35 - DNA, 109 bp, Artificial
  Synthetic DNA
agatctggggaaggttcagcagctcgaaccaaggggtggaatagtataccattcgtggtatagtatcccacccgggtttgggtttgggtttgggtttttttttaagctt

SEQ ID NO: 36 - DNA, 112 bp, Artificial
  Synthetic DNA
agatctggggaaggttcagcagctcgaaccaaggggtggaatagtataccattcgtggtatagtatcccaccctctagagggccctgaagagggccctttttttttaagctt

SEQ ID NO: 37 - DNA, 136 bp, Artificial
  Synthetic DNA
agatctgggagagggccctgaagagggcccttttctgaaggttcagcagctcgaaccaaggggtggaatagtataccattcgtggtatagtatcccacccgggtttgggtttgggtttgggtttttttttaagctt

SEQ ID NO: 38 - DNA, 143 bp, Artificial
  Synthetic DNA
agatctgggtgctcgcttcggcagcacatatactagtcgacgaaggttcagcagctcgaaccaaggggtggaatagtataccattcgtggtatagtatcccaccctctagagcggacttcggtccgcttttttttttaagctt

SEQ ID NO: 39 - DNA, 253 bp, Artificial
  Synthetic DNA
gaattcgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacagatct

SEQ ID NO: 40 - DNA, 516 bp, Artificial
  Synthetic DNA
cacgtcgtgcctcacatcgagcccgtggctagatgcatcatccctgatctgatcggaatgggtaagtccggcaagagcgggaatggctcatatcgcctcctggatcactacaagtacctcaccgcttagttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtc

SEQ ID NO: 41 - DNA, 18 bp, Artificial
  Synthetic DNA
ggtaacgctgcctccagc

SEQ ID NO: 42 - DNA, 20 bp, Artificial
  Synthetic DNA
tgtagttgcggacaatctgg

SEQ ID NO: 43 - DNA, 20 bp, Artificial
  Synthetic DNA
ctcgctgcaagcaaatgaac

SEQ ID NO: 44 - DNA, 20 bp, Artificial
  Synthetic DNA
tcgtcccaggactcgatcac

SEQ ID NO: 45 - DNA, 104 bp, Artificial
  Synthetic DNA
agatcgggtcttcatggccttgtgcagccgctgggtggttcgccacctcagtcctggaaacagggaccgggtttgggtttgggtttgggtttttttttaagctt

SEQ ID NO: 46 - DNA, 107 bp, Artificial
  Synthetic DNA
agatcgggtcttcatggccttgtgcagccgctgggtggttcgccacctcagtcctggaaacagggacctctagagggccctgaagagggccctttttttttaagctt

SEQ ID NO: 47 - DNA, 138 bp, Artificial
  Synthetic DNA
agatcgggtgctcgcttcggcagcacatatactagtcgactcttcatggccttgtgcagccgctgggtggttcgccacctcagtcctggaaacagggacctctagagcggacttcggtccgcttttttttttaagctt

SEQ ID NO: 48 - DNA, 84 bp, Artificial
  Synthetic DNA
agatcgggtcttcatggccttgtgcagccgctgggtggaatagtataccattcgtggtatagtatcccacccttttttaagctt

SEQ ID NO: 49 - DNA, 80 bp, Artificial
  Synthetic DNA
agatcgggtcttcatggccttgtgcagccgctgggtggttcgccacctcagtcctggaaacagggaccttttttaagctt

SEQ ID NO: 50 - DNA, 60 bp, Artificial
  Synthetic DNA
agatcgggtcttcatggccttgtgcagccgctgggtggttcgccacctttttttaagctt

SEQ ID NO: 51 - DNA, 25 bp, Artificial
  Synthetic DNA
ggccgatgctaagaacattaagaag

SEQ ID NO: 52 - DNA, 20 bp, Artificial
  Synthetic DNA
ccacggtaggctgagaaatg

SEQ ID NO: 53 - DNA, 18 bp, Artificial
  Synthetic DNA
ctccgatgaacagggcgc